(19) United States
(12) Patent Application Publication
(10) Pub. No.: US 2013/0203631 A1
(43) Pub. Date: Aug. 8, 2013

(54) FRAMELESS MULTIPLEXED MICROARRAYS

(71) Applicant: PRIMORIGEN BIOSCIENCES, LLC, Madison, WI (US)

(72) Inventors: Thomas J. Burke, Madison, WI (US); William L. Patterson, Stoughton, WI (US); Tobias C. Zutz, Madison, WI (US)

(73) Assignee: PRIMORIGEN BIOSCIENCES, LLC, Madison, WI (US)

(21) Appl. No.: 13/826,538

(22) Filed: Mar. 14, 2013

Related U.S. Application Data
(63) Continuation of application No. 13/204,089, filed on Aug. 5, 2011, now abandoned.
Application No. 13/204,089 is a continuation of application No. 12/233,140, filed on Sep. 18, 2008, now abandoned.
(60) Provisional application No. 60/994,179, filed on Sep. 18, 2007.

(51) Int. Cl. G01N 33/543

(52) U.S. Cl. CPC G01N 33/543
USPC 506/16; 506/30; 506/18

(57) ABSTRACT

The present invention relates to novel methods for the quantitative detection of molecules in an array. In particular, the present invention relates to methods and apparatuses for producing a frameless array. In another embodiment, the present invention relates to a composition comprising nitrocellulose that is useful of producing a frameless array. In another embodiment, the present invention relates to a method for detecting a molecular interaction. In yet another embodiment, the present invention relates to kits useful for practicing the methods and apparatuses of the present invention. The present invention provides improved methods and apparatuses for the high throughput analysis of molecular interactions and quantitative detection.

### CROSS-REFERENCE TO RELATED APPLICATION

This application is a continuation application of U.S. patent application Ser. No. 13/204,089 filed Aug. 5, 2011, which is a continuation application of U.S. patent application Ser. No. 12/233,140 filed Sep. 18, 2008, which is a non-provisional application of U.S. Provisional Application No. 60/994,179, filed Sep. 18, 2007. This application and the above-referenced applications claim priority to U.S. Provisional Application No. 60/994,179, filed Sep. 18, 2007 all of which are incorporated by reference in their entireties herein.

### FIELD OF THE INVENTION

The present invention relates to novel assay methods and compositions for the quantitative detection of molecules. In one aspect, the invention relates to a method for producing a frameless array that is useful for the quantitative detection of molecules. In another aspect, the present invention relates to a method of detecting molecular interactions. In yet another aspect, the invention relates to a device useful for the detection of molecular interactions. In still another aspect, the invention relates to kits for the detection of molecular interactions.

### BACKGROUND OF THE INVENTION

### Proteins:

Proteins facilitate many of the cell's most basic functions of reproduction, metabolism, growth, and programmed death. The study of proteins has contributed to our understanding of almost all cellular operations including transcription, replication, translation, splicing, secretion, cell-cycle control, signal transduction, and cellular architecture. Since the advent of cellular biology there has been a systematic analysis of protein presence and structure and function, which has increased understanding of how proteins and protein complexes function in the cell. In the past few years, thousands of new protein sequences and post-translational variants have been identified by the human genome and proteome projects. This protein jackpot has hastened the drive to understand protein-based regulation but it also challenges researchers to find better biophysical methods to quantitatively detect protein markers. Better multiplexed, quantitative, high throughput methods will help us understand the correlation between protein markers and cellular functions. There is demand for inexpensive, miniaturized, multiplexed, high throughput assays that can generate thousands of protein detection data points per experiment.

Proteins are linear polymers of amino acids; the primary sequence is defined by the actual sequence of the 20 amino acids. Post-translational modifications such as phosphorylation, methylation, acetylation, amidation, and glycosylation create very large numbers of protein variants. Other protein modification mechanisms such as ubiquitinylation, sumoylation, or ISGylation, create a branched a variety of protein structures. Proteins may exhibit localized folding (secondary structure) in parts of the amino acid polymer such as alpha helixes or beta sheets. The tertiary structure of a protein is the folding pattern throughout the whole molecule, it defines the three-dimensional shape. Protein function is related to its tertiary structure and this relationship is a core tenet of structural biology. An isolated protein is said to be in its native conformation when it retains the same tertiary structure inside and outside the cell.

### Assays:

Multiplexed protein assays, where multiple analytes can be detected per sample, have been developed in several formats, including: (1) microbeads, (2) arrays on planar surfaces, (3) arrays on three-dimensional surfaces such as nitrocellulose and hydrogels and (4) arrays in microplate wells. Each of these formats has disadvantages for specific applications. Some of the common disadvantages include the high cost of assay equipment and reagents, the limitations on the number of analytes that can be multiplexed, the inability to perform multiplexing in a high throughput mode, and the customization time and expense for developing each assay. Some assay methods are suitable for large proteome projects dealing with small sample numbers studying thousands of different proteins while other methods are more suited for dealing with only dozens of proteins from thousands of samples.

Many detection modes have been incorporated into protein arrays including colorimetric, fluorescent, radioactive and label-free methods. The choice of detection method depends on the statistical quality required (accuracy, precision, limit of quantitation, etc.), the background in the test sample, and the availability of affordable instrumentation to build the assay and perform the analytical measurements.

One of the most common protein detections assays is the immunoassay, which can be configured in a multitude of ways. The three most common formats are: (1) the single antibody array; (2) the dual (matched) antibody array; and (3) the antigen array. The single antibody and the antigen arrays can be multiplexed with hundreds of analytes but vary in specificity, sensitivity, and quantitativeness. Often these assays are designed as semi-quantitative (e.g., ratiometric). Dual antibody assays can give high quality, quantitative data but are limited in multiplexing by the number of matched antibody pairs that can be identified. Because of the time involved in screening for matched antibody pairs with current methods, dual antibody assays can be expensive to develop.

In one form of immunoassay, the sandwich ELISA, a capture antibody, which captures the analyte, is bound to a solid surface and the surface is blocked with a non-specific reagent. A sample is added that contains the analyte, the captured analyte is washed with buffer and a second (detection) antibody is added that recognizes a portion of the analyte, which is distinct from the binding site of the first antibody. The second antibody is then detected directly or indirectly by a variety of methods. In another form of the immunoassay, a capture antibody is attached to a surface. Next, the surface is blocked with a non-specific reagent, and a sample is added that contains a labeled analyte. Finally, the captured analyte is washed with a buffer and the labeled analyte is detected directly or indirectly. In yet another form of the immunoassay, an analyte is attached to surface and blocked with a non-specific reagent. A sample containing an antibody, such as serum, is then added. Next, the captured antibody is washed with buffer, and the antibody is detected directly or indirectly.

In some assays, the detectable moieties can be positioned on many components of the assay, including the analyte. For example, fluorescence assays can be designed so that one or more assay components are fluorescently labeled and various fluorescent properties are measured. These include assays involving fluorescence intensity, fluorescence lifetime, fluorescence resonance energy transfer, fluorescence polarization (anisotropy), and time-resolved fluorescence. The dynamic range of an immunoassay can be >1000 and the detection limit varies, but a common lower limit for protein detection is approximately 1-10 pg/ml of analyte. Immunoassays have been modified with different sample extraction protocols and many different natural and synthetic surfaces have been utilized. Other modes of detection are reviewed in Reviews in Fluorescence 2004 (Chris D. Geddes and Joseph R. Lakowicz).

Immunoassays often are used to detect proteins from a variety of sources including viruses, prions, bacteria, fungi, and plant or animal fluids, cells, or tissues. The source of the protein is not limited for immunoassays but in many cases, the protein is extracted and partially purified before it can be used. Many different extraction procedures have been developed, which include physical methods such as freeze-thaw cycling, sonication, high temperature or high pressure (French Press) treatment, or glass bead vortexing. Other methods employ chemical or biochemical methods, such as detergent disruption, enzymatic lysis, or creating a strongly reducing environment. Commonly, extraction methods incorporate a combination of both physical and chemical treatments. After the initial treatment, a separation step is commonly employed such as centrifugation, magnetic particle separations, phase separations, or precipitation reactions to further clarify the sample for detection.

There are several limitations to all multiplexed arrays, whether they are on beads, in microplate wells or on planar or three-dimensional slides. For arrays in microplate wells, it is difficult to find a robust arraying method that can deposit a high number of protein spots in a timely manner into the bottom of the well. The array detection method in microplate wells is often by chemiluminescence; therefore the spots cannot be spaced as closely together as in fluorescence detection. In 96 well microplates, the large sample volumes can also be a limiting factor. The 384 well microplate requires less sample but fewer analytes can be arrayed in the well bottom. For all multiplexed immunoassays, finding antibodies that show acceptable sensitivity and specificity without cross-reacting with other antibodies is a significant and expensive challenge.

A large number of protein spots can be arrayed on array slides but it is necessary to attach a well former (frame) to each slide to keep the samples separate (see FIG. 1). Typically, a 16 well frame is attached to a standard (25 mm×75 mm) glass slide. In essence, this creates a temporary 16 well microplate out of an arrayed slide. The frame must seal tightly so there is no leakage among the wells. The use of a removable well-forming frame limits the type of protein binding material that can be attached to the substrate glass. If the protein binding material is too thick and porous, a tight seal will not develop between the wells. Conversely, thin layers of protein binding material can provide a tight seal but are often limited by lower protein binding capacity. In addition, the frame must also be removed before scanning or imaging the slide, which can often be challenging as it is important to ensure that the material holding the immobilized protein is not removed with the frame.

Bead-based detection systems have been developed to allow analysis of several analytes simultaneously. The multiplexed bead array format commercialized by LUMINEX (Luminex Corp., Austin, Tex.), called xMAP system uses antibody-coated colored latex particles to capture analytes, which then are detected by a second labeled antibody. Each uniquely colored bead has a different capture antibody allowing mixtures of several beads. The particles are directed through a flow cytometer that identifies the particle based on the bead color (fluorescence) and measures the fluorescence of the detection antibody associated with that bead. One drawback to this method is that the capture antibodies sometimes are inactivated when they are covalently immobilized to the latex beads. In addition, the assay still requires a significant, sometime excessive, sample volume. This system also is limited in that the number of assays that can be multiplexed is only a few dozen.

### Detection Methods:

Three common methods for detecting proteins on surfaces are ELISA (enzyme-linked immunosorbent assay), FIA (fluorescence immunoassay), and SPR (surface plasmon resonance). Colorimetric detection in an ELISA uses an enzyme, such as alkaline phosphatase or horseradish peroxidase that is conjugated to the detection antibody and use colorimetric enzyme substrates. These conjugated enzymes can also use chemiluminescent substrates. SPR does not require protein labeling but it does require protein immobilization; it is a suitable technique for direct capture antibody or antigen assays. However, the technique has not been sufficiently developed for multiplexed microarrays.

Fluorescence detection has been the mainstay of the DNA array technology and there are several commercialized instruments that can detect fluorescence from very small arrayed spots on a glass slide or other surface. Most of the instruments have 2 fluorescence excitation lasers, typically 532 nm and 635 nm, intended for the excitation (and detection) of Cyanine 3 (Cy3) and Cyanine 5 (Cy5) fluorescent labels. These instruments also can scan slides for fluorescent, quantitative protein detection. In addition, new instruments are being introduced that not only detect higher wavelength dyes (there is less fluorescence background from biological samples and surfaces at higher wavelengths), but also new plate formats, such as 75×125 mm plates.

Novel fluorescent labels and labeling techniques are being introduced for protein arrays. Molecular Probes/Invitrogen (Carlsbad, Calif.) has numerous fluorescent dyes with various reactive groups for labeling proteins, labeling secondary antibodies, and labeling affinity binding proteins (e.g., streptavidin). In addition, they have very small (submicron) fluorescent latex particles that can be linked to proteins for affinity binding. Dyomics GmbH (Jena, Germany), in collaboration with Pierce Biotechnology (Rockford, Ill.), has developed its own line of reactive highly fluorescent dyes that range into the infrared wavelengths (700 to 800 nm). LI-COR Biosciences (Omaha, Nebr.) has developed infrared dyes to correspond with its imaging systems (ODYSSEY and AERIUS) that are focused on in vivo methodologies. Protein-based fluorescent molecules, such as R-phycoerythrin and phycobilisomes (SENSILIGHT Dye from Martek Biosciences, Columbia, Md.), also are used in the protein microarray arena. Also, several novel detection methods have been developed, such as the rolling circle amplification (RCA).

### Surfaces:

Nitrocellulose (cellulose nitrate) has been used for protein and nucleic acid binding experiments for decades, demonstrating its versatility, robustness, and affordability. Proteins, including antibodies, placed directly on hydrophobic surfaces such as glass or plastic will partially denature, reducing protein activity. However, the porous, polymeric features of nitrocellulose allow binding through hydrophobic interactions, hydrogen bonding, and Van der Waals interactions that minimally disrupt the protein. Proteins can be spotted (dot blots) or transferred from a polyacrylamide gel electrophoresis (PAGE), as typically performed in a Western blot.

In addition to its use in life science research and diagnostic assays, nitrocellulose has been used as a component in explosives, photography papers, paints and lacquers, and ink for inkjet printers. Due to this widespread use, it continues to be developed and better characterized as a raw material, especially for analytical purposes. Many different grades of nitrocellulose can be purchased based on purity, nitrogen content, viscosity (molecular weight), solvents, wetting agents, phlegmatizers, and plasticizers. Two common suppliers are Wolff Cellulosics (Walsrode Industrial Park) and Filo Chemicals (New York, N.Y.); many other suppliers exist worldwide.

For diagnostic and life science research applications, nitrocellulose is commonly referred to as a “nitrocellulose membrane” and is used in two forms: a white stand alone layer of nitrocellulose or a coating on a surface, usually glass. The fluorescence background of a white, porous nitrocellulose surface is typically significantly higher than a thin optically clear nitrocellulose surface. The coated surface ranges from thick (>10 μm) white, porous coating such as slides from Schleicher and Schull (Whatman, Middlesex, UK) and Grace BioLabs (Bend, Oreg.) to an optically clear, ultra-thin coating (<500 nm) slides from GenTel Biosciences (Madison, Wis.). The physical properties of applied nitrocellulose such as thickness, porosity, hydrophobicity, strength, adhesiveness, homogeneity, and protein binding capacity are determined by a large number of factors. These include the ratio of solvents, co-solvents and non-solvents, the drying conditions including temperature, humidity, and solvent partial pressures, and the presence of other molecules such as plasticizers, stabilizers, and other cellulose esters.

Another use of nitrocellulose spots is in matrix assisted laser desorption/ionization time-of-flight mass spectrometry (MALDI-TOF MS) (Luque-Garcia et al, Anal. Chem., 78 (14), 5102-5108, 2006; Zhao X, et al, Analyst 129(9):817-822, 2004). In this example, an array of 50 clear nitrocellulose spots (500 μM) were mixed with α-cyano-4-hydroxycinnamic acid (CHCA) matrix solution and then peptide samples were deposited on top of the nitrocellulose on a stainless steel MALDI plate. The samples were then ionized by a laser for mass spectrometry analysis.

Nitrocellulose can also be applied to substrate surfaces such as silicon and various plastics such as polyethylene terephthalate, cellulose acetate, polycarbonate, or polystyrene. A wide range of organic solvents can be used to adhere nitrocellulose to polymeric surfaces including acetone, methanol, ethanol, propanol, isopropanol, methylene chloride, n-butanol, and methyl ethyl ketone. The choice of solvents in coating plastics with nitrocellulose must take into account both the adhesive properties (strength) but also the physical properties of the bound nitrocellulose. For instance, in U.S. Patent Application Publication 2000/0160120 A1, several solvents mixtures were tested for their ability to adhere nitrocellulose to plastic. In this application, the bonded nitrocellulose was used as an adhesive to adhere an additional polymer to the plastic surface. Three solvents, ethanol, methylene chloride, and methyl ethyl ketone, were demonstrated to bind nitrocellulose to plastic but the net result was a lacquer-like adhesive. The resulting product did not provide a porous layer for absorbing protein. The selection of solvents optimized for both binding nitrocellulose to plastic and producing a coating adequate for protein binding was not addressed.

The solvents used for dissolving nitrocellulose and coating surfaces fall into three groups (Wolff-cellkulosics.com). True (or active solvents) completely dissolve nitrocellulose at room temperature. These include ketones (acetone, methyl ethyl ketone, methyl isobutyl ketone), esters (ethyl acetate, butyl acetate, methoxy propyl acetate) and glycol ethers (methyl glycol ether, ethyl glycol ether, isopropyl glycol ether). Latent solvents cannot dissolve nitrocellulose at room temperature. When mixed with some true solvents or certain non-solvents they become capable of dissolving nitrocellulose. Examples include alcohols (ethanol, isopropanol, and butanol) and ethers (diethyl ether). Non solvents cannot dissolve nitrocellulose directly or indirectly. These include aliphatic and aromatic hydrocarbons (benzenes, toluene, and xylene). The judicious selection of solvents for creating nitrocellulose surfaces also depends on the solubility of nitrocellulose/cellulose acetate mixtures, which are a common formulation used in membranes and surface coatings.

Grace BioLabs (Bend, Oreg.) manufactures a glass slide coated with nitrocellulose that is distributed mainly by Schleicher & Schuell, now owned by Whatman (Middlesex, UK). The surface of these slides is relatively thick at ≈14 microns compared to many surface chemistries (<0.2 □micron). This surface has high protein binding capacity, which is important for tissue and lysate arrays, but has very high fluorescence background that reduces its utility for highly sensitive detection. Recently, GenTel Biosciences (Madison, Wis.) introduced a thin-film (<0.5 □micron) nitrocellulose coated glass slide (licensed from Clinical MicroArrays, now called Decision Biomarkers, Natick, Mass.). Other companies, Agnitio Science & Technology (Taiwan) and PriTest (Redmond, Wash.) have also produced nitrocellulose coated slides for protein detection.

The slide from the above-mentioned manufacturers is designed to be used with a removable silicone gasket (frame, well former) that creates multiwells to isolate specimens and reagents and to prevent cross contamination. In the absence of a frame to create the wells, solutions that are applied to the nitrocellulose areas easily flow across the slide and fail to remain isolated. However, it is often difficult and challenging to remove the frame without removing the material holding the specimens. Grace slides and accessories are available through many other companies such as Interchim (Interchim.com (France), Stratech (Suffolk, England), and Invitrogen (Carlsbad, Calif.).

Nitrocellulose deposition methods on surfaces, such as spraying (atomization), dip coating, or pipetting solutions, determine both the thickness and the physical properties of the final product. Most coatings cover the entire surface but in some cases, dots or islands of nitrocellulose are deposited on sections of the glass surface. Grace BioLabs (Bend, Oreg.) has produced the ONCYTE film-wells for cell based microarrays in which multiple nitrocellulose dots are affixed to a glass microscope slide. However, the area on the slide between the white dots is not described as hydrophobic and when tested, this area does not demonstrate hydrophobicity. The lack of hydrophobicity between the dots contributes to cross-contamination of samples, and reduces assay throughput and accuracy.

Therefore, the need still exists for a method of generating a solid surface substrate useful for the quantitative detection of molecules in an array that provides minimal cross-contamination and high-throughput analysis. Frameless arrays and methods for producing and using such arrays, which are independent of well formers or framers, would be extremely useful.

### SUMMARY OF THE INVENTION

The present invention relates to methods and apparatuses for producing a frameless array. The present invention can be applied to many different depositions of membranes for many types of assays. In one embodiment, a solid surface or substrate for the quantitative detection of molecules is generated without the need for frames or wells to separate samples.

In another embodiment, the present invention relates to a method for producing a frameless array comprising: coupling at least two segregated membranes to a substrate, wherein said membranes comprise a composition comprising nitrocellulose, and further wherein said composition is formulated to maintain an applied sample on the membrane; and coupling an analyte to said membranes to produce a frameless array. In another embodiment, the composition is formulated to allow an applied sample to cover the entire membrane. In yet another embodiment, the composition further comprises cellulose acetate and a solvent.

In another embodiment, the present invention relates to a frameless array comprising: (a) at least two segregated membranes coupled to a substrate, wherein said membranes comprise a composition comprising nitrocellulose, and further wherein said composition is formulated to maintain an applied fluid within the perimeter of the membrane and (b) an analyte coupled to said membranes. In yet another embodiment, the analyte is selected from the group consisting of: a probe, RNA, DNA, a peptide, an extract, a fragment of a protein, an antibody, and a protein.

In still yet another embodiment, the present invention relates to a method for detecting a molecular interaction comprising: (a) applying a sample to an array comprising at least two segregated membranes coupled to a substrate, wherein said membranes comprise a composition comprising nitrocellulose and an analyte coupled to said membrane, and further wherein said composition is formulated to maintain an applied fluid within the perimeter of the membrane; and (b) detecting a molecular interaction.

In another embodiment, the present invention relates to a method for producing an array comprising: dispensing at least two segregated membranes to a substrate, wherein said membranes comprise a composition comprising nitrocellulose, wherein said composition is formulated to maintain an applied fluid within the perimeter of the membrane; and coupling an analyte to said membrane.

In another embodiment, the present invention relates to a method for producing an array comprising: dispensing at least two segregated membranes to a substrate, wherein said membranes comprise a composition comprising nitrocellulose, wherein said composition is formulated to maintain an applied fluid within the perimeter of the membrane; and coupling an analyte to said membrane.

In another embodiment, the present invention relates to a kit comprising: a frameless array, wherein said array comprises at least two segregated membranes; reagents for performing molecular detection of a molecule and instructions for using said array and said reagents.

In another embodiment, the present invention relates to a kit comprising a frameless array, wherein said array comprises at least two segregated membranes and further wherein said membranes are arrayed with an analyte; reagents for performing molecular detection of said molecule and instructions for using said array and said reagents

In yet another embodiment, the present invention relates to a method for producing a frameless array comprising: (a) a substrate; (b) a hydrophobic layer coated on the substrate; and (c) a membrane applied to an area of the substrate coated with the hydrophobic layer, wherein said membrane comprises a composition comprising nitrocellulose, and further wherein said composition is formulated to maintain a sample within the perimeter of the membrane.

In another embodiment, membranes may be coupled directly to a plastic surface. Separate assays can be performed on each membrane. The different membranes can be treated with different blocking agents, analyte solutions, or detection reagents creating versatility in assay optimization or analyte detection. Multiplexed assays can be performed simultaneously on 96 or 384 hydrophilic sections on a single plastic surface.

In yet another embodiment, the assays performed on the membranes are immunoassays. The detection methods for the immunoassays include but are not limited to fluorescent assays and colorimetric assays.

### BRIEF DESCRIPTION OF THE DRAWINGS

FIG. 1 is a photograph of an exemplary removable 16-well frame (well former) that is commonly used for slide arrays.

FIG. 2 is a schematic of an exemplary array design using nitrocellulose sections (dots) on hydrophobic surface. In the schematic, which is provided for demonstration purposes only, the left assay schematic is an antigen array using colorimetric detection and the right assay is a multiplexed sandwich ELISA using fluorescence detection.

FIG. 3 contains three photographs showing various arrays with varying numbers of membranes or dots on each array. FIG. 3A is a photograph of an array comprising 96 nitrocellulose membranes (dots) on a hydrophobic glass surface. FIG. 3B is a photograph of an array comprising 96 nitrocellulose membranes (dots) on a plastic surface. FIG. 3C is a photograph of an array depicting 384 nitrocellulose membranes (spots) on a plastic surface. Each nitrocellulose membrane can be arrayed with analytes.

FIG. 4 contains two photographs each showing an array with various membranes or dots and various numbers of analytes arrayed on each membrane. FIG. 4A is a photograph of an antigen array on part of a glass slide showing four nitrocellulose membranes with 6 arrayed areas of protein on each membrane. FIG. 4B is a photograph of a 96 membrane array with 9 protein areas on each nitrocellulose membrane (dots). Two membranes (dots) are enlarged to the right of the 96 membrane array.

FIG. 5 is a line graph reporting colorimetric ELISA detection of Interleukin 4 (IL-4) and Interleukin 10 (IL-10) using the nitrocellulose frameless array.

FIG. 6 is a schematic of an antigen array depicting near infrared fluorescent detection of mouse IgG.

FIG. 7 is a bar graph reporting the results of a protein concentration measurement assay using protein attached to nitrocellulose membranes (dots) and near infrared fluorescent dyes.

FIG. 8 is a bar graph reporting the capacity of nitrocellulose membranes (dots) with Mouse IgG Spots.

FIG. 9 is a bar graph reporting the direct, colorimetric detection of binding between S-adenosyl homocysteine conjugated to carrier proteins and antibodies in mouse hybridoma supernatants.

FIG. 10 is a photograph depicting protein detection (100 μM) on a nitrocellulose surface.

FIG. 11 is a line graph reporting colorimetric sandwich ELISA detection of Sox2 proteins and specificity testing using the frameless array.

FIG. 12 is photograph of the frameless array in a perpendicular (vertical) position showing that an applied fluid sample to each membrane remains in position even when the array is turned sideways. The smaller photograph is an enlargement of a single sample on a membrane.

FIG. 13 is a photograph of a frameless array membrane comprising transparent nitrocellulose. A sample was placed on the transparent nitrocellulose membrane and the array was turned sideways.

FIG. 14 is a photograph demonstrating protein detection using transparent nitrocellulose arrays.

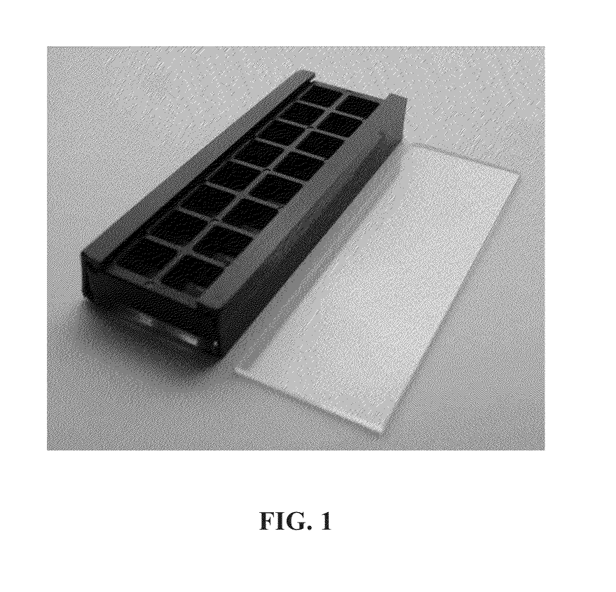

The objects and advantages of the invention will appear more fully from the following detailed description of the preferred embodiment of the invention made in conjunction with the accompanying drawings.

### DETAILED DESCRIPTION OF THE INVENTION

### Definitions

To facilitate an understanding of the present invention, a number of terms and phrases are defined below:

“Antibody mimetic” means a molecule that replicates essential features of an immunoglobulin, monoclonal or polyclonal antibody.

“Assay” and like terms means a procedure for detecting the presence, estimating the concentration, and determining the biological activity of a macromolecule, molecule, ion, or cell. Assays are based on measurable parameters that enable the evaluation of differences between samples and controls.

“Multiplex assay” means a procedure for the parallel analysis of samples.

“Serum” means the cell-free portion of the blood from which the fibrinogen has been separated in the process of clotting. The cell free portion of the blood (plasma) has a pH within the narrow range of 7.35 to 7.45 in healthy individuals.

“Sample” means a specimen or culture obtained from any source, as well as biological and environmental samples. Biological samples may be obtained from animals (including humans) and encompass fluids, solids, tissues, and gases. Biological samples include urine and blood products, such as plasma, serum and the like. Such examples are not however to be construed as limiting the sample types applicable to the present invention.

“Immunoglobulin” or “Antibody” means a protein that binds a specific antigen. Immunoglobulins include, but are not limited to, polyclonal, monoclonal, chimeric, and humanized antibodies, Fab fragments, F(ab′)₂ fragments, including immunoglobulins of the following classes: IgG, IgA, IgM, IgD, IgE, and secreted immunoglobulins (slg). Immunoglobulins generally comprise two identical heavy chains and two light chains. However, the terms “antibody” and “immunoglobulin” also encompass single chain antibodies and two chain antibodies.

“Analyte” means a substance being measured or a substance used to measure another substance in an analytical procedure.

“Antigen” means a substance capable, under appropriate conditions, of inducing a specific immune response and of reacting with the products of that response, which in preferred embodiments is a specific antibody. Antigens may be soluble substances, such as toxins and foreign proteins, or particulate, such as bacteria and tissue cells, however, only the portion of the antigen molecule known as the antigenic determinant or epitope combines with antibody.

“Specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

“Non-specific binding” and “background binding” when used in reference to the interaction of an antibody and a protein or peptide means an interaction that is not dependent on the presence of a particular structure (i.e., the antibody is binding to proteins in general rather that a particular structure such as an epitope).

“Label,” “marker” and “reporter” mean any atom or molecule that can be used to provide a detectable (preferably quantifiable) signal. Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral.

“Instructions for using said kit” refers to instructions for using the reagents contained in the kit including but not limited to instructions for the detection of analyte in a sample from a subject. In some embodiments, the instructions further comprise the statement of intended use required by the U.S. Food and Drug Administration (FDA) in labeling in vitro diagnostic products.

“Subject” means any animal (e.g., a mammal), including, but not limited to, humans, non-human primates, rodents, and the like, which is to be the recipient of a particular diagnostic test or treatment. Typically, the terms “subject” and “patient” are used interchangeably herein in reference to a human subject.

“Non-human animals” means all non-human animals including, but are not limited to, vertebrates such as rodents, non-human primates, ovines, bovines, ruminants, lagomorphs, porcines, caprines, equines, canines, felines, ayes, etc.

“Amino acid sequence” and terms such as “polypeptide” or “protein” are not meant to limit the amino acid sequence to the complete, native amino acid sequence associated with the recited protein molecule.

“Humidity chamber” means a closed chamber at room temperature with >60% relative humidity, unless stated otherwise.

“Solid surface” means any solid surface suitable for the attachment of biological molecules and the performance of molecular interaction assays. Surfaces may be made of any suitable material (e.g., including, but not limited to, metal, glass, and plastic) and may be modified with coatings (e.g., a metal, a polymer or an epoxy).

“Substrate” refers to any material with a surface that may be coated with a film.

“Coated with a film” in regard to a substrate mean a situation where at least a portion of a substrate surface has a film arrayed on it (e.g., through covalent or non-covalent attachment).

“Microarray” means a solid surface comprising a plurality of addressed biological macromolecules (e.g., nucleic acids or antibodies). The location of each of the macromolecules in the microarray is known, so as to allow for identification of the samples following analysis.

“Array of first proteins” means a microarray of polypeptides on a solid support.

“Biological macromolecule” means large molecules (e.g., polymers) typically found in living organisms. Examples include, but are not limited to, proteins, nucleic acids, lipids, and carbohydrates.

“Target molecule” means a molecule in a sample to be detected. Examples of target molecules include, but are not limited to, oligonucleotides (e.g., containing a particular DNA binding domain recognition sequence), viruses, polypeptides, antibodies, naturally occurring drugs, synthetic drugs, pollutants, allergens, affector molecules, growth factors, chemokines, cytokines, and lymphokines.

“Binding partners” means two molecules (e.g., proteins) that are capable of, or suspected of being capable of, physically interacting with each other. As used herein, the terms “first binding partner” and “second binding partner” refer to two binding partners that are capable of, or suspected of being capable of, physically interacting with each other.

The term “wherein said second binding partner is capable of interacting with said first binding partner” refers to first and second binding partners that are known, or are suspected of being able to interact. The interaction may be any covalent or non-covalent (e.g., hydrophobic or hydrogen bond) interaction.

“Signal” means any detectable effect, such as would be caused or provided by an assay reaction. For example, in some embodiments of the present invention, signals are SPR or fluorescent signals. In other embodiments, the presence of an RNA synthesized from a gene of interest is the signal.

“Gene” means a nucleic acid (e.g., DNA) sequence that comprises coding sequences necessary for the production of a polypeptide, RNA (e.g., including but not limited to, mRNA, tRNA and rRNA) or precursor (e.g., precursors). The polypeptide, RNA, or precursor can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

“Hydrophilic membrane,” and “hydrophilic section” mean any material that is wettable with water, and includes but is not limited to a film, a coating, a segregated section of a larger substrate, a composition that is wettable with water; a composition that when dried is wettable with water; a solution that is wettable with water; a solution that when dried is wettable with water; membranes that comprise one or a plurality of components containing hydrophilic material, such as nitrocellulose, regenerated cellulose or polysulfone, which is hydrophilized with polyvinylpyrrolidone (PVP). Additional suitable membrane materials are polyacrylonitrile, cellulose fibers (for example, as available under the trade designation Cuprophan from Akzo, Netherlands), cellulose acetate, and the like. As alternatives to membranes already comprising hydrophilic material, hydrophobic membranes can also be used if they have been made hydrophilic with hydrophilizing agents, which can be washed out, such as myristyl alcohol or with a water/ethanol mixture.

“Segregated membrane” means a membrane that is set apart or separated from another membrane.

### Methods for Producing a Frameless Array

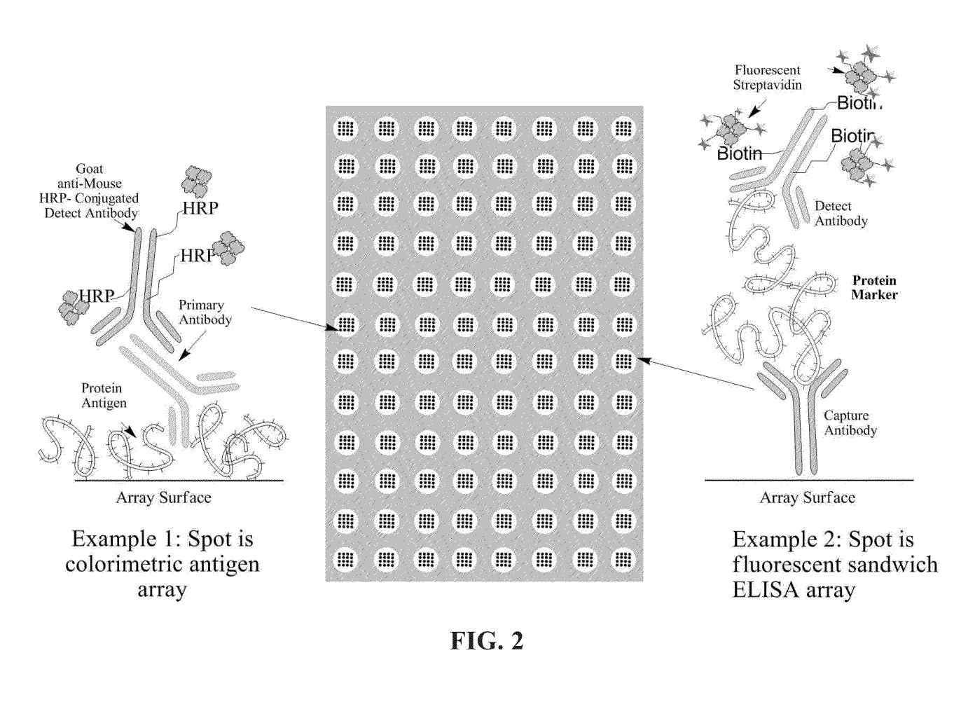

A significant challenge in the detection of molecular interactions with microarrays is to find a method that is both high throughput and utilizes multiplexed assays. In one aspect, the present invention provides a method for generating a solid surface that can be used to produce tens of thousands of detection data points in a single day (FIG. 2).

In one embodiment, the present invention relates to a method for producing a frameless array comprising: affixing a hydrophilic membrane to a hydrophobic surface on a solid substrate and arraying the hydrophilic membrane with an analyte for detection. The hydrophobic surface may be pre-existing or it can be generated. In yet another embodiment, the present invention relates to a method for producing a frameless array comprising: coupling at least two segregated membranes to a substrate, wherein said membranes comprise a composition comprising nitrocellulose, and further wherein said composition is formulated to maintain an applied sample within the perimeter of the membrane; and attaching an analyte on said membranes to produce a frameless array. In yet another embodiment, the present invention relates to a method of producing a frameless array without the need for well-framers. In still another embodiment, the present invention relates to a method for producing a frameless array without the need for hydrophobic ink.

1. Solid Surface or Substrate

In one embodiment, the solid surface or substrate includes but is not limited to the use of supports comprising glass, cellulose acetate, a metal, polypropylene, teflon, polyethylene, polyester, polypropylene, polycarbonate, polyethylene terephthalate, polystyrene, and ceramics. Glass in the meaning of the invention comprises materials in amorphous, non-crystalline solid state, i.e., the glassy state in the meaning of the invention can be regarded as frozen, subcooled liquid or melt. Thus, glass materials are inorganic or organic, mostly oxide melted products converted into a solid state by an introduction process without crystallization of the melt phase components. Crystals, melts, and subcooled melts are also to be regarded as glass materials in the meaning of the invention. For example, glass materials can be flat glass, container glass, commercial glass, laboratory glass, lead glass, fiber glass, optical fiber glass, and others. It is also possible to use glass materials free of silicate, e.g., phosphate glass materials.

Metals also include metallic glasses, i.e., materials being in a metastable, largely amorphous state. Polymers having metallic conductivity are also included in the meaning of the invention. Polypropylenes in the meaning of the invention are thermoplastic polymers of propylene. Polypropylenes are remarkable particularly for their high hardness, resilience, rigidity, and heat resistance. Teflon is a polytetrafluoroethylene, which advantageously has good thermoplastic properties. Polyethylenes are completely inert when exposed to water, alkaline solutions, salt solutions and inorganic acids. For example, supports comprising polyethylenes have a very low water vapor permeability.

Polyesters are compounds produced by ring-opening polymerization of lactones or by polycondensation of hydroxycarboxylic acids or of diols and dicarboxylic acids or dicarboxylic acid derivatives. Polyesters also comprise polyester resins, polyester imides, polyester rubbers, polyesterpolyols, and polyesterpolyurethanes. Polyesters are thermoplastics and have distinct material character. They have high thermal stability and can be processed into alloys with metals such as copper, aluminum and magnesium. Ceramics is a collective term for an especially inorganic class of materials predominantly consisting of non-metallic compounds and elements and particularly comprising more than 30% by volume of crystalline materials. Various ceramics or ceramic materials include but are not limited to pottery, earthenware crockery, split wall tiles, laboratory porcelain, crockery porcelain, bone china, aluminum oxide ceramics, permanent magnet materials, silica bricks, and magnesia bricks can be concerned. Clay-ceramic materials are classified in coarse and fine materials, with fine clay-ceramic materials comprising earthenware, stoneware and porcelain.

In another embodiment, the substrate can be any size and thickness including but not limited to 0.1-1 mm, 1-5 mm, 5-10, and 10-15 mm. In still yet another embodiment, the substrate can be a single, flat layer without dividers or individual wells.

2. Hydrophobic Surface

The solid surface or substrate can comprise a hydrophobic surface or can be treated with a solution to create a hydrophobic surface. Any solution or compound that creates a hydrophobic surface when applied to the solid surface can be used including but not limited to methyl and octyl derivates, reactive epoxides and epoxy adhesives. The solution or compound can be applied to the solid surface in any manner that creates the hydrophobic surface including but not limited to dipping the solid substrate into the solution or compound, spraying the solution onto the solid substrate, spreading the compound onto the solid substrate, and pippeting the solution on the solid substrate.

3. Hydrophilic Membrane/“Membrane”

In one embodiment, absorptive membranes are coupled to a solid surface. In yet another embodiment, the hydrophilic membranes are coupled to a solid surface comprising a hydrophobic surface. The isolated hydrophilic membranes can be coupled to the solid surface without the need for frames (well framers or well formers). The hydrophobic area between the hydrophilic membrane demonstrates strong protein binding capacity, thus if sample leaches off a hydrophilic section, the protein will not contaminate an adjacent section. The absorptive hydrophilic membranes are designed so that as the analyte-containing samples are slowly dispensed, they are absorbed by the hydrophilic sections in real time.

Hydrophilic membranes include but are not limited to nitrocellulose, polyvinylidene difluoride (PVDF), cellulose acetate, organic cellulose esters (also know as gun cotton), cellulose mixed esters, polytetrafluoroethylene (PTFE), polyamide, regenerated cellulose, polycarbonate, polystyrene, polypropylene, polyterephthalate, polyester, polysulfone, polyacrylamide, agarose, nylon, polyprene, and mixtures of nitrocellulose and cellulose acetate. Membranes requiring pre-wetting as well as membranes that do not require pre-wetting may be used. Membranes of the present invention include but are not limited to compositions that are wetable with water, compositions that when dried are wetable with water, solutions that are wetable with water, and solutions that when dried are wetable with water.

Nitrocelluloses are inorganic cellulose esters. Any type of nitrocellulose can be used including but not limited to white, transparent, opaque, translucent, nitrocellulose in powder form, and nitrocellulose in liquid form. Any size or shape of nitrocellulose can be used. White nitrocellulose or transparent nitrocellulose can be used or a combination of white and transparent. Protran® is a nitrocellulose membrane commercially available from Whatman. Westran S is made of PVDF is also available from Whatman. The nitrocellulose may be obtained in a powder form and then dissolved in the appropriate solution or the nitrocellulose may be obtained already in solution.

Solvents can be used for dissolving the hydrophilic membrane including but not limited to nitrocellulose and compositions of nitrocellulose and cellulose acetate. True (or active solvents) can be used and typically dissolve nitrocellulose at room temperature. These include but are not limited to ketones (acetone, methyl ethyl ketone, methyl isobutyl ketone), esters (ethyl acetate, butyl acetate, methoxy propyl acetate) and glycol ethers (methyl glycol ether, ethyl glycol ether, isopropyl glycol ether). Latent solvents also can be used to dissolve the hydrophilic membrane. In general, latent solvents cannot dissolve nitrocellulose at room temperature. When mixed with some true solvents or certain non-solvents they become capable of dissolving nitrocellulose. Examples include but are not limited to alcohols (ethanol, isopropanol, and butanol) and ethers (diethyl ether). The judicious selection of solvents for creating nitrocellulose surfaces also depends on the solubility of nitrocellulose/cellulose acetate mixtures, which are a common formulation used in membranes and surface coatings.

In yet another embodiment, segregated membranes can be coupled to a substrate. In still another embodiment, segregated membranes can comprise a composition comprising nitrocellulose. In yet another embodiment, the composition can comprise nitrocellulose, cellulose acetate and a solvent. In still another embodiment, the composition can comprise a single solvent or more than one solvent. In another embodiment, the solvent can be selected from the group consisting of acetone, ethanol, amyl acetate, butanol and more than one solvent. In yet another embodiment, the composition comprises the solvents acetone, ethanol and butanol. In still another embodiment, the solvents acetone, butanol and ethanol comprise greater than 80% of the solvent.

In yet another embodiment, any number of segregated membranes can be coupled to a substrate including but not limited to 2-7,8,9-11, 12, 13-15, 16, 17-23, 24, 25-35, 36, 37-47, 48, 49-95, 96, 97-383, 384, 385-1535, 1536, 1537-6133, 6144, and greater than 6144. In one embodiment, 96 membranes can be coupled to the substrate in an 8×12 grid, with about 9 millimeters apart. In another embodiment, 384 membranes can be coupled to a substrate in a 16×24 grid, with 4.5 millimeters apart. In still another embodiment, 1536 membranes can be coupled to a substrate in a 32×48 grid, with 2.25 millimeters apart.

In yet another embodiment, each membrane can comprise any area adequate for the task including but not limited to 0.25-0.5 square microns, 0.5-1.0 square microns, 1.0-1.5 square microns, 1.5-2.0 square microns, 2.0-2.5 square microns, 2.5-5.0 square microns, 5-10 square microns, 10-20 square microns, 20-40 square microns, 40-100 square microns, 0.1-0.5 square millimeters, 0.5-1 square millimeters, 1-5 square millimeters, 5-10 square millimeters, 10-15 square millimeters, 15-20 square millimeters, 20-25 square millimeters, 25-50 square millimeters, 50-100 square millimeters, 100-200 square millimeters, and greater than 200 square millimeters. In still yet another embodiment, the area can be selected from the group consisting of 1, 7, and 28 square millimeters.

In another embodiment, each membrane can be any size appropriate for the task including but not limited to a circle, a square, a rectangle, a triangle, an octagon, oval, pentagon, hexagon, parallelogram, rhombus, kite, and trapezium. In another embodiment, the array can comprise can comprise membranes coupled to the substrate of all the same shape and size, the array can comprise membranes of coupled to the substrate of more than one shape and the array can comprise membranes coupled to the substrate of more than one size.

In still another embodiment, the present invention relates to segregated membranes, which are coupled to a substrate, comprising a composition, wherein said composition is formulated to maintain an applied fluid within the perimeter of the membrane. In another embodiment, the present invention relates to a composition formulated to maintain a fluid within the perimeter of a membrane for a period of time selected from the group consisting of 0.1-0.5, 0.51-1.0, 1.1-2.0, 2.1-4.0, 4.1-6.0, 6.1-8, 8.1-10, 10.1-12, 12.1-16, 16.1-20, 20.1-24, 24.1-30, 30.1-36, 36.1-48, 48.1-54, 54.1-60, 60.1-72, 72-1-96, and 96.1-120 hours. In still yet another embodiment, the composition is formulated to allow the applied solution to cover the entire segregated membrane and maintain the applied fluid within the perimeter of the membrane.

In yet another embodiment, the composition can comprise a percentage of nitrocellulose ranging from 0.1% to 10%. In another embodiment, the composition can comprise a percentage of cellulose acetate ranging from 0.03% to 3%. In still another embodiment, the composition can comprise a solvent mixture (by volume) comprising: 48-54% acetone; 32-38% ethanol; and 10-20% n-butanol.

In still another embodiment, it is possible to use nylon, with nylon in the meaning of the invention comprising linear aliphatic polyamides. Polyvinylidene fluorides may also be used, which are thermoplastics that are easy to process and advantageously, have a high resistance when exposed to temperature and chemicals. In another embodiment, cellulose acetate may be used.

The hydrophilic membrane can be applied to the solid surface or substrate by any means that allows the hydrophilic membrane to retain its ability to interact with molecules. A formulation comprising the hydrophilic membrane and other reagents may be created to aid in the attachment of the hydrophilic membrane to the solid surface or substrate. Any formulation comprising a hydrophilic membrane may be used provided that the formulation provides stable binding to solid surfaces as well as optimal protein binding. The formulation can be obtained by dissolving the hydrophilic membrane into a solvent. Reagents useful for creation of the formulations include but are not limited to amyl acetate, methanol, acetone, ethyl acetate, ethanol, isopropanol, water, n-butanol, diethyl ether, glycerol, ethylene glycol, and cellulose acetate.

The formulations can be optimized for solubility, clarity and porosity of the hydrophilic membrane, ease of pipetting the sample, stability of the sample, and ease of scaling up production. Some parameters to consider when testing and creating the formulations are: (1) order of addition of solvents; (2) solvent ratios in the mixtures; (3) solvent concentration; (4) porosity of the final coating; (5) evenness of coating by the hydrophilic membrane; (6) ratio of one hydrophilic membrane to another hydrophilic membrane; (7) background fluorescence of the coating; and (8) stability of binding to a solid surface-even in the long term presence of aqueous detergents.

The hydrophilic membrane or formulation comprising the hydrophilic membrane can be applied to the hydrophobic surface or substrate using any method that allows for molecular interactions with the hydrophilic membrane including but not limited to pipetting, dispensing, spraying, atomizing, layering, and spreading.

In one embodiment, the formulation comprising the hydrophilic membrane can be sprayed onto the solid substrate. The formulation comprising the hydrophilic membrane can be atomized using an ultrasonic spraying device (ultrasonic nozzle). For example, a formulation can be made comprising nitrocellulose. As used herein, a nitrocellulose solution is a solution that contains between 0.1% weight/volume and 99.9% weight/volume nitrocellulose. The solution may comprise other compounds or biological macromolecules provided that the amount of nitrocellulose in the solution is in the previously defined range. The ultrasonic spraying device includes a hydrophilic membrane solution container and a spraying nozzle that is communicatively extended from the hydrophilic membrane solution container. The ultrasonic spraying nozzle atomizes the hydrophilic membrane solution in order to apply an even spray of hydrophilic membrane particles on the solid substrate. Exemplary ultrasonic spraying nozzles are commercially available from Sono-Tek Corporation (Milton, N.Y.). Exemplary Sono-Tek models include the 8700-25, 8700-35, 8700-48, 8700-48H, 8700-60, 8700-120, and 8600-6015.

In another embodiment, any type of nebulizer (atomizer) can be used to atomize the hydrophilic membrane. In some embodiments, the atomizing device comprises a nebulizer in which a hydrophilic membrane solution is guided to flow through a tube by a high-pressure stream of gas. In some embodiments, the nebulizer is air-assisted using a gas such as nitrogen in order to control a flow rate of the hydrophilic membrane particles at the nebulizer so as to control the thickness of the hydrophilic membrane film on the solid substrate.

In another embodiment, the segregated membranes can be coupled to the substrate by dispensing a composition comprising nitrocellulose. The composition can be dispensed using any machine suitable for the task including but not limited to the Nanodrop I, Nanodrop ExtY, the Nanodrop II, Nanodrop Express, the Screenmaker 96+8, and the Platemaker HTS, all available from Innovadyne Technologies (Santa Rosa, Calif.).

In another embodiment, the segregated membranes can be coupled to a plastic substrate. In yet another embodiment, the segregated membranes comprise a composition comprising nitrocellulose. In still another embodiment, the plastic substrate includes but is not limited to PEI cellulose.

In yet another embodiment, the present invention relates to a method for producing a frameless array comprising dispensing a composition comprising nitrocellulose onto a polyester film; and drying said film in a humidity chamber. In still another embodiment, the composition further comprises cellulose acetate and a solvent. In still another embodiment, the humidity chamber is greater than 60% relative humidity.

4. Attaching an analyte on a Membrane

Any analyte can be attached on a membrane including but not limited to a probe, an antibody, a molecule, a small molecule inhibitor, an antibody, a peptide, a peptide mimetic, fragment of a protein, active region of a protein, a protein, amino acid sequence, single stranded nucleic acid, RNA, DNA, and a fragment of a gene. In yet another embodiment, any number of analytes can be attached to each membrane including but not 1-5,6-10, 11-15, 16-20, 21-25, 26-30, 31-40, 41-50, 51-100, and greater than 100. In still yet another embodiment, the same analyte or a different analyte can be attached to each membrane. In another embodiment, each membrane of the array can be arrayed with the same analyte, the same set of analytes or different analytes.

In yet another embodiment, each analyte coupled to the membrane can have an individual area selected from the group consisting of: 1-10, 11-20, 21-30, 31-40, 41-50, 51-60, 61-70, 71-74, 75, 76-100, 101-149, 150, 151-200, 201-250, 251-300, 301-350, 351-400, 401-449, 450, 451-500, 501-749, 750, 751-1000, and greater than 1000 microns in diameter.

### Frameless Array

In another embodiment, the present invention relates to a frameless array comprising: at least two segregated membranes coupled to a substrate, wherein said membranes comprise a composition comprising nitrocellulose. In yet another embodiment, the composition is formulated to maintain an applied fluid within the perimeter of the membrane. In still yet another embodiment, the top of the composition is the highest point on the frameless array. In another embodiment, an analyte is attached to said membranes

In yet another embodiment, the frameless array can be used to detect an analyte without the need for well-framers, well-formers or hydrophobic ink. The frameless arrays of the present invention can be used in the absence of well-framers, well-formers or hydrophobic ink.

In still yet another embodiment, the frameless array can be used to detect any analyte of interest including but not limited a probe, a molecule, a small molecule inhibitor, an antibody, a peptide, a peptide mimetic, fragment of a protein, active region of a protein, a protein, amino acid sequence, single stranded nucleic acid, RNA, DNA, and a fragment of a gene.

In another embodiment, the invention relates to a frameless array that allows the deposition and containment of individual fluid samples on segregated membranes, wherein the membranes comprise a composition comprising nitrocellulose, and also allows subsequent reactions of the membranes to be performed in a single vessel at one time, thereby eliminating the need to perform reactions in each individual well. The frameless array of the present invention can be used to detect analytes by performing reactions in a single vessel. The frameless array of the present invention eliminates the need to perform each reaction in a single well of a plate, thereby eliminating well-to-well variation, and improving the sensitivity of the assay.

The following is an example of an application of the frameless array and should not be construed to limit the embodiments of the present invention. Ninety-six segregated membranes can be coupled to a substrate, which is a single layer substrate without dividers or individual wells. Nine antibodies of interest can be attached to each membrane. A distinct cell extract can be applied to each membrane. From this point forward, all reaction for the 96 membranes can be carried out as a single step including but not limited to rinsing, applying a secondary antibody, washing and the reagents and processes necessary for detecting the presence of an analyte, which in this case refers to the nine antibodies.

In still yet another embodiment, the frameless array can be used to detect analytes on multiple occasions. Portions or sections of the frameless array can be easily separated such that an array comprising 96 membranes can be separated into individual membranes or into a group of membranes that includes any number of membranes including but not limited to 1-7, 8, 9-11, 12, 13-23, 24, 25-47, 48, 49-59, 60, and 61-95. The frameless array of the present invention allows the membranes to be separated into appropriate numbers. For example, an array may comprise 96 membranes with 9 distinct antibodies on each membrane. If there are only 24 samples, the array can be cut into four sections, with each section comprising 24 membranes. Samples then can be applied to the twenty-four membranes and the remaining three sections (24 membranes per section) can be stored for use on a future date. In addition, the frameless arrays of the present invention provide for easy storage, and can be stored as a single membrane or as a group of membranes.

### Method for Detecting Molecular Interactions

In yet another embodiment, the present invention relates to a method for detecting a molecular interaction comprising: (a) applying a sample to an array comprising at least two segregated membranes coupled to a substrate, wherein said membranes comprise a composition comprising nitrocellulose, and an analyte attached to said membrane; and (b) detecting a molecular interaction. In another embodiment, the composition is formulated to maintain an applied fluid within the perimeter of the membrane.

In another embodiments, the sample can be obtained from a source selected from the group consisting of: a bacterium, prion, fungus, virus, plant, protozoan, animal, human, non-human, mammal, reptile, cattle, cat, dog, goat, swine, pig, monkey, ape, gorilla, bull, cow, bear, horse, sheep, poultry, mouse, rat, fish, dolphin, whale, or shark.

In still another embodiment, the sample can be a cell, a cell extract, a plant extract, lectin, tissue, organ blood, serum, plasma, saliva, urine, tear, vaginal secretion, sweat, umbilical cord blood, chorionic villi, amniotic fluid, embryonic tissue, an embryo, a two-celled embryo, a four-celled embryo, an eight celled embryo, a 16-celled embryo, lymph fluid, cerebrospinal fluid, semen, mucosa secretion, peritoneal fluid, sputum, respiratory exudates, ascitic fluid, mucosa secretion, peritoneal fluid fecal matter, or body exudates. The sample can be purified or can represent a lysate at any state of purification from a tissue or organ. The sample can be a whole cell lysate including but not limited to NIH293, A-20, HeLa, HepG2, Jurkat, PC-3, SW480, T24, U937, and WI-38 whole cell lysate. The sample can be a subcellular fraction cell lysate including but not limited to a cytoplasmic protein lysate, a membrane protein lysate, and a nuclear protein lysate. In addition, the sample can be a cell extract at any stage of purification including but not limited to an extract that represent merely disrupting the cell, an extract that involves one purification step, and an extract that involves more than one purification step. The cell extract can be obtained from specific types of cells including cancer cells, hybridomas, liver, kidney, bladder, ovary, adipose tissue, lymph node, cervix, pancreas, brain, lung, heart, spleen, thyroid, breast, colon, and prostate cells.

In another embodiments, the sample can be applied in any appropriate volume including but not limited to 1-5,6-10, 11-20, 21-30, 31-50, 51-100, 101-200, 201-300, 301-500, 501-1000 microliters. One of ordinary skill in the art will understand that the appropriate sample volume to be applied is proportional to the size of the membrane. In still yet another embodiment, the sample is applied in a volume to cover each segregated membrane.

In another embodiment, the methods and apparatus of the present invention can be used to detect any analyte including but not limited to a probe, a molecule, a small molecule inhibitor, a protein, a fragment of a protein, an active region of a protein, a peptide, a peptide mimetic, and an amino acid sequence, RNA, DNA, a single stranded nucleic acid including but not limited to an oligonucleotide and primer, and double stranded nucleic acids. The nucleic acid that is to be analyzed can be any nucleic acid, e.g., genomic, plasmid, cosmid, yeast artificial chromosomes, artificial or man-made DNA, including unique DNA sequences, and also DNA that has been reverse transcribed from an RNA sample, such as cDNA. The nucleic acid can comprise a single nucleotide polymorphism (SNP), a mutation, or more than one mutation. Oligonucleotide probes and primers of any length can be used to detect nucleic acids.

In another embodiment, the method of detection can be any suitable method including but not limited to colorimetric, fluorescent, near infrared fluorescent, ultraviolet spectrometry, silver deposition, chemiluminescent, ELISA, and electrochemiluminescent.

### Kits:

The methods of the invention are most conveniently practiced by providing the reagents used in the methods in the form of kits. In one embodiment, the present invention relates to a kit comprising: a frameless array, wherein said array comprises at least two segregated membranes; reagents for performing molecular detection of a molecule and instructions for using said array and said reagents. In yet another embodiment, the segregated membranes comprise a composition comprising nitrocellulose. In still yet another embodiment, the composition is formulated to maintain an applied fluid within the perimeter of the membrane.

In another embodiment, the present invention relates to a kit comprising a frameless array, wherein said array comprises at least two segregated membranes and further wherein a molecule is attached to the membranes; reagents for performing molecular detection of said molecule and instructions for using said array and said reagents.

A kit preferably contains one or more of the following components: written instructions for the use of the kit, appropriate buffers, salts, a solid substrate, a hydrophobic solution or compound, such as an epoxy, and a hydrophilic membrane, such as nitrocellulose, detergents, and if desired, water of the appropriate purity, confined in separate containers or packages, such components allowing the user of the kit to create a solid surface useful for quantitative detection of molecular interactions. The kit may also contain antibodies, labeled antibodies, oligonucleotides, primers, controls and other useful reagents for detection of molecules. The primers that are provided with the kit will vary, depending upon the purpose of the kit and the DNA that is desired to be tested using the kit.

A kit can also be designed to detect a desired or variety of molecular interactions, especially those associated with an undesired condition or disease. For example, one kit can comprise, among other components, a set or sets of antibodies to detect proteins associated with breast cancer. Another kit can comprise, among other components, a set or sets of antibodies to detect colon cancer. Still, another kit can comprise, among other components, a set or sets of primers for genes associated with a predisposition to develop heart disease.

The following examples illustrate various embodiments of the invention, but should not be construed to limit the scope of the invention in any manner.

### SPECIFIC EMBODIMENTS

### Example 1

### Preparation of Glass Coated with Epoxy Adhesive and Affixing Nitrocellulose

Standard microscope slides were purchased from (Fisher Scientific, Chicago, Ill.) and cleaned by autoclaving 45 minutes at (240° F.) in a 1-5% solution of Cascade (Proctor and Gamble) detergent. The slides were rinsed multiple times in deionized water to remove all residual detergent and were dried in a clean sterile hood. In some cases, the slides were dried rapidly in a 300° F.-350° F. oven for 5-10 minutes. Alternatively, pre-cleaned slides can be obtained from Erie Scientific, Portsmouth, N.H.

Each slide was treated by dip coating (dipped once) in a diluted epoxy adhesive manufactured by Henkel Consumer Adhesives (Avon, Ohio). The adhesive contained silica quartz (40-60%), aliphatic amine (10-20%), benzoyl alcohol (5-10%), silica fumed (5-10%), formaldehyde polymer with toluene (5-10%), Phenol 2,4,6 tris[(dimethylamino) methyl] (5-10%), N-isotridecyloxypropyl-trimethylene diamine (1-5%), propylene glycol (1-5%), and isophoronediamine (1-5%). The epoxy adhesive was prepared as per the manufacturer's instructions and diluted 10 fold in acetone. (Fisher Scientific, Pittsburgh, Pa.). The diluted mixture was centrifuged for 20 minutes at 15000×g and the material above the silica pellet was removed for dip coating the slides. Each slide was dipped 1-4 seconds and dried immediately in airflow of approximately 400 feet per second. The dried slides were stored at room temperature.

Alternatively, the clean slides were coated with an epoxy adhesive from Environmental Technologies, (Fields Landing, Calif.). The resin components were nonyl phenol, and polyoxyalkyleneamines and the hardener components were bisphenol A/epichlorohydrin resin and C12 and C14 alkyl glcidyl ethers. The exact concentrations of the components are considered confidential for the manufacturer, Environmental Technologies. In the preferred embodiment, equal volumes of the hardener and resin for the clear casting epoxy were mixed and diluted 16 fold in amyl acetate (Fisher Scientific, Pittsburgh, Pa.). Approximately 400 μl of the solution was pipetted to the level surface of a clean 25×75 mm glass slide. The slide was placed in a small closed container at room temperature and allowed to dry slowly over approximately 1 hour. If the coated slide was dried too rapidly, the coating was uneven and unacceptable for nitrocellulose binding. This epoxy adhesive provided an optically clear surface on the glass. The dried slides were stored at room temperature in a closed container. Other solvents such as butanol and isopropanol may be substituted for amyl acetate but the more volatile the solvent, the more rigorously the drying process must be controlled in order to allow polymerization of the epoxy adhesive.

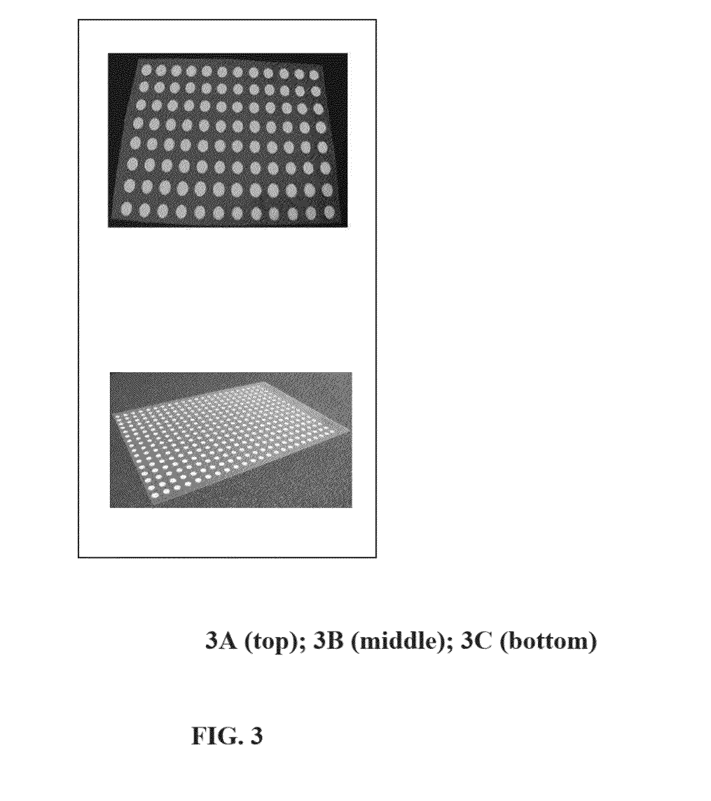

Nitrocellulose was purchase as a 10% solution in acetone from Ladd Research (Williston, Vt.). Cellulose acetate was purchased for Sigma Chemical (St. Louis, Mo.) and dissolved in acetone. A nitrocellulose mixture was prepared: 3% nitrocellulose, 0.3% cellulose acetate, with a final solvent concentration (by volume) of 51% acetone, 35% ethanol, and 14% n-butanol. To cover an entire slide, 500-600 p. 1 was pipetted to the surface of a level slide and dried rapidly in a clean environment. For creating nitrocellulose dots in a 96 (8×12) grid on a surface (FIG. 3A), approximately 14 μl of the solution was pipetted using a Beckman Biomek pipetting station directly on to the surface. Alternatively, the nitrocellulose solution can be pipetted (dispensed) directly on to polyester films and dried in >65% relative humidity with rapid air movement (FIG. 3B). For creating a 384 well grid (24×16), approximately 1.8 μl of the solution was pipetted on to the surface (FIG. 3C).

Alternatively, nitrocellulose solutions in amyl acetate can be dispensed directly to glass or plastic substrates to produce a transparent version of the frameless array. Ethyl acetate may be substituted for the acetone as it is less volatile and easier to dispense.

### Example 2

### Colorimetric Detection of Mouse IgG on Nitrocellulose Membranes (Dots)

Six individual membranes (dots) were printed with the same concentration of mouse IgG (Equitech-Bio, Kerrville Tex.) using the Calligrapher Arrayer (Bio-Rad, Hercules Calif.) with solid pins. The spots were allowed to dry slowly in high humidity for 15 minutes and then each membrane was blocked with 20 μL of a non-protein block solution (Pierce Biosciences, Rockford Ill.) for one hour in a humidity chamber. The excess block solution was aspirated off and the membranes (dots) were allowed to dry. A goat anti-mouse IgG horse radish peroxidase (HRP) labeled antibody (Santa Cruz Biotechnology, Santa Cruz Calif.) was diluted to 1 μg/mL in PBST buffer and used to cover the entire slide. The slide was then washed with phosphate buffered saline plus Tween 20 solution for one hour and rinsed several times with deionized ultra pure water. The slide was incubated with a TMB (3,3′,5,5′-tetramethylbenzidene) stabilized substrate (Promega Corporation, Madison Wis.) until the background intensity began to appear. Then, the slide was rinsed several times with the deionized ultra pure water and placed in a particle free hood to dry.

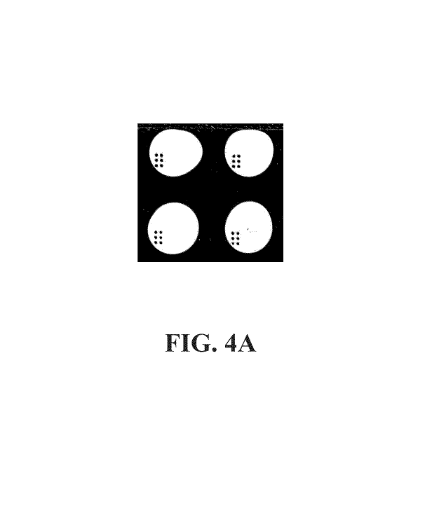

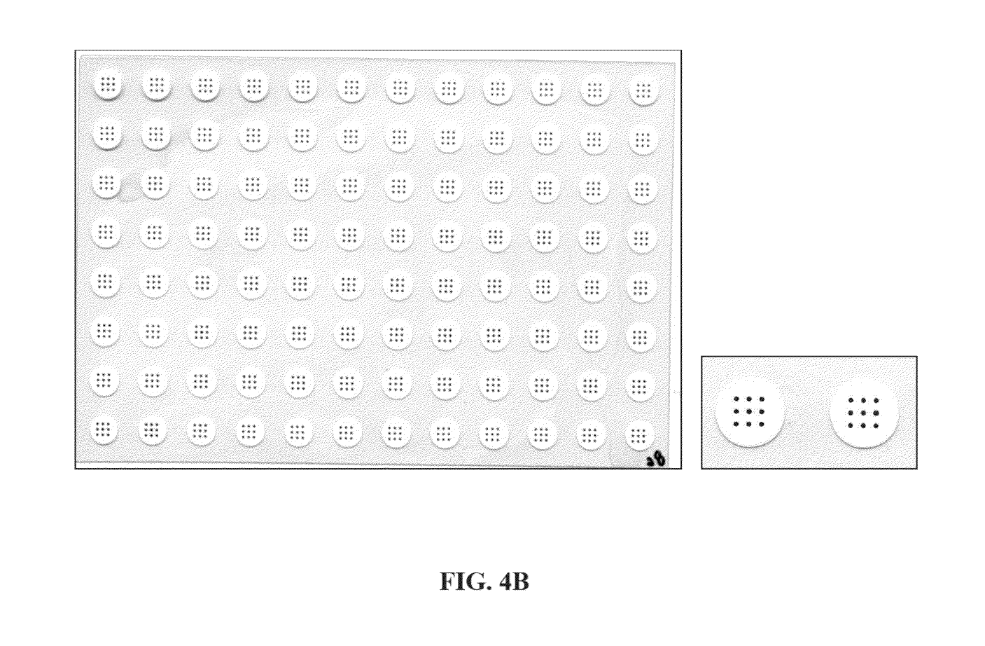

The dried slide was scanned with a High Resolution scanner (Epson America, Long Beach, Calif.) at 2400 dpi in 16-bit grayscale file (FIG. 4A). The image was inverted using Photoshop Elements (Adobe Systems Inc., San Jose, Calif.) and saved as a TIFF file labeled with an additional “INVERTED.” The image was analyzed with the GenePix Pro 6.1 software (Molecular Devices, Sunnyvale, Calif.) and interpreted with Microsoft Excel spreadsheet software. (Microsoft Corporation, Redmond, Wash.)

In a separate reaction, mouse IgG (Jackson ImmunoResearch Laboratories, West Grove, Pa.) was diluted to 200 μg/ml in phosphate buffered saline and printed using the Calligrapher Arrayer (Bio-Rad, Hercules, Calif.) in a 3 by 3 grid by a solid pin on all 96 membranes (dots) of a pre-equilibrated nitrocellulose array. After printing, the sheet was blocked with 1×NAP Block (GBiosciences, Maryland Heights, Mo.) in phosphate buffered saline with 0.05% Tween 20 (PBST) for 1 hour at room temperature. It was washed with PBST 3 times and then incubated with 15 mL of a 1:1000 dilution of anti-mouse IgG alkaline phosphatase (Jackson ImmunoResearch Laboratories) at room temperature for one hour. It was washed again with PBST 5 times, rinsed with water, and incubated with BCIP/NBT substrate (Moss Inc., Pasadena, Md.) until signal could be seen, ˜5 minutes. The sheet was rinsed with water and allowed to dry. The array was scanned with a high resolution Epson V700 scanner (Epson America, Long Beach, Calif.) at 1200 dpi in 16-bit grayscale TIFF file (FIG. 4B). The Epson software was used to automatically determine the Histogram Adjustment Parameters. The image was inverted using Photoshop Elements (Adobe Systems Inc., San Jose, Calif.) and saved as a TIFF file. The image was analyzed with the GenePix Pro 6.1 software (Molecular Devices, Sunnyvale, Calif.) and interpreted with Microsoft Excel spreadsheet software. (Microsoft Corporation, Redmond, Wash.). Table I provides the average colorimetric intensity value and relevant statistics of all 96 membranes. Table II provides the average colorimetric intensity for each column and the relevant statistics. Table III provides the average colorimetric intensity for each row and the relevant statistics.

TABLE I
Quantitative Array Analysis of Mouse IgG Spotted to 96
Nitrocellulose Membranes

| | |
|---|---|
| Average colorimetric intensity of all 864 spots | 55,513 |
| (96 dots × 9 spots) | |
| Standard Deviation | 2,167 |
| Coefficient of Variation | 3.90% |

TABLE II
Quantitative Array Analysis of Mouse IgG Spotted to 96
Nitrocellulose Dots-By Column

| | Colorimetric | Standard | % Coefficient |
|---|---|---|---|
| Column | Intensity | Deviation | of Variation |
| 1 | 57764.6 | 2253.4 | 3.90% |
| 2 | 54535.7 | 2457.6 | 4.50% |
| 3 | 54606.4 | 1874.2 | 3.40% |
| 4 | 55383.6 | 1928.1 | 3.50% |
| 5 | 55856.7 | 2143.5 | 3.80% |
| 6 | 55903.9 | 2070.1 | 3.70% |
| 7 | 55431.2 | 1655.1 | 3.00% |
| 8 | 55995.0 | 1516.2 | 2.70% |
| 9 | 55976.4 | 1712.7 | 3.10% |
| 10 | 56062.4 | 1921.7 | 3.40% |
| 11 | 54090.4 | 1949.1 | 3.60% |
| 12 | 54551.2 | 1839.4 | 3.40% |

TABLE III
Quantitative Array Analysis of Mouse IgG Spotted to 96
Nitrocellulose Dots-By Row

| | Colorimetric | Standard | % Coefficient |
|---|---|---|---|
| Row | Intensity | Deviation | of Variation |
| 1 | 53151.5 | 2098.6 | 3.9 |
| 2 | 55924.2 | 1843.2 | 3.3 |
| 3 | 55799.7 | 1458.0 | 2.6 |
| 4 | 54663.7 | 1990.5 | 3.6 |
| 5 | 55246.1 | 1991.8 | 3.6 |
| 6 | 56491.1 | 1918.8 | 3.4 |
| 7 | 56587.1 | 1893.6 | 3.3 |
| 8 | 56241.6 | 1802.7 | 3.2 |

### Example 3

### Colorimetric ELISA Detection of Interleukin 10 (IL-10) and Interleukin 4 (IL 4) Protein on Nitrocellulose Membranes (Dots)

An Elisa using colorimetric detection was performed on nitrocellulose membranes (dots). The slides were made as per Example 1.

1. Primary Printing of Capture Antibodies

Two different capture antibodies against IL-4, and IL-10 (EBiosciences, San Diego, Calif.) were printed in quadruplicate using a Calligrapher Arrayer (BioRad, Hercules, Calif.). All arrayed antibodies were printed at a concentration of 100 μg/mL on each of 24 different nitrocellulose membranes (dots) as prepared as in Example 1. The antibodies were printed in 1× phosphate buffered saline (PBS) and the slide was allowed to dry in high humidity for 20 minutes. Each nitrocellulose dot was blocked with 20 μL of the Pierce non-protein block (Pierce Biosciences, Rockford, Ill.) in a humidity chamber for 1 hour. The blocking buffer was then aspirated off the surface.

2. Addition of Antigens (Cytokines)

A dilution series of the IL-4 and IL-10 antigens (EBiosciences, San Diego, Calif.) was prepared beginning with a stock solution of 5 ng/mL (5,000 pg/ml) stock. This stock was serially diluted 11 times at a ratio of 1:1 to give 12 different concentrations of the antigen diluted in PBS. A representation of the protein concentrations on the slide is shown below in Table IV. Each concentration in the table represents the concentration of the interleukin analyte on each of the corresponding nitrocellulose membranes (dots).

TABLE IV
Concentration of antigen on each respective nitrocellulose
membrane (dot).

| Nitrocellulose | Concentration of |
|---|---|
| Membrane | Antigen (pg/mL) |
| 1 | 2.4 |
| 2 | 4.9 |
| 3 | 9.8 |
| 4 | 20 |
| 5 | 39 |
| 6 | 78 |
| 7 | 156 |
| 8 | 313 |
| 9 | 625 |
| 10 | 1250 |
| 11 | 2500 |
| 12 | 5000 |

The slide was incubated in a high humidity chamber for 1 hour and the solutions and the slides washed by immersion into phosphate buffered saline containing 0.05% Tween 20 detergent (PBST buffer). The slide was washed 4 times with the same wash solution.

3. Addition of Detect Antibodies, Avidin-Horse Radish Peroxidase (HRP), and Stabilized TMB Substrate

IL-4 and IL-10 biotinylated antibodies (EBioscience, San Diego, Calif.) were each diluted 1:250 in PBST buffer. Four milliliters of the dilutent were incubated on the arrayed slides for one hour at room temperature in a humidity chamber. The slide was washed 4 times with PBST, and 3 times with deionized, ultra pure water. The slide was then incubated for 30 minutes at room temperature with 4 mL of the avidin-HRP (Pierce Bioscience, Rockford, Ill.) diluted 1 to 250 in PBST. The slide was again washed with PBST (4 times) and ultra pure water (3 times) and then incubated with enough TMB stabilized substrate (Promega Corporation, Madison Wis.) to cover the slide. The HRP catalysis of the TMB substrate produces a blue/violet precipitate on the white nitrocellulose membranes (dots). The catalysis was allowed to continue 5-120 minutes, depending on the sensitivity that was needed in the assay: the development was monitored visually. When the background began to develop, the slide was washed several times with deionized water and it was allowed to dry in a sterile, clean hood.

4. Documentation and Analysis of the Slide

The dried slide was scanned with a High Resolution scanner (Epson America, Long Beach, Calif.) at 2400 dpi in 16-bit grayscale file. The image was inverted using Photoshop Elements (Adobe Systems Inc., San Jose, Calif.) and saved as a TIFF file labeled with an additional “INVERTED.” The image was analyzed with the GenePix Pro 6.0 software (Molecular Devices, Sunnyvale, Calif.) and interpreted with Microsoft Excel spreadsheet software. (Microsoft Corporation, Redmond, Wash.)

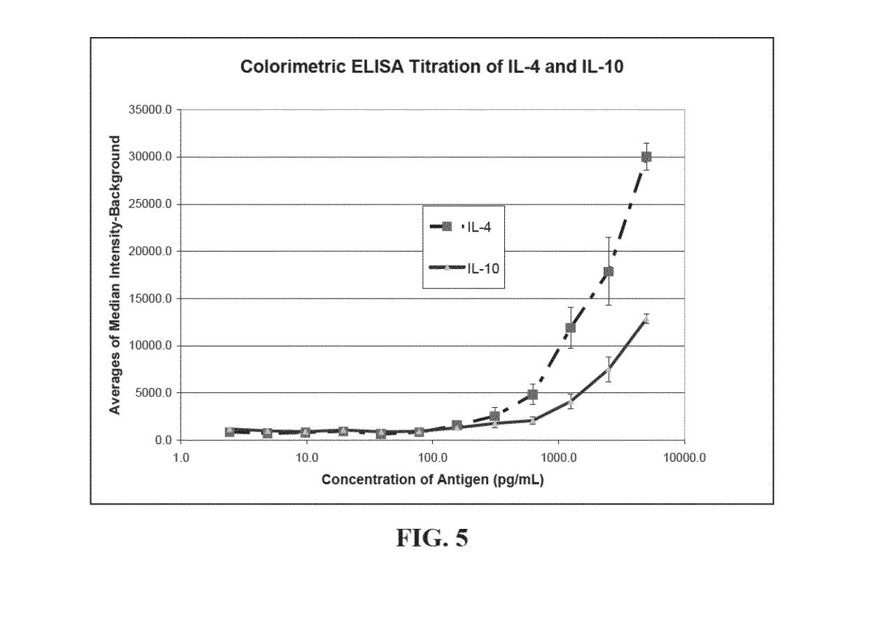

As the concentration of antigen increased, the intensity of the signal also increased (FIG. 5). This demonstrates that the antigen-antibody binding was specific, and that antigen bound to the nitrocellulose was in an active confirmation suitable for interaction with the appropriate antibody. It also demonstrates that the sandwich ELISA assay is capable of detecting analyte in the picograms/milliliter of sample. Optimization could produce assays that can detect 1-10 picograms of analyte per milliliter.

### Example 4

### Use of Near Infrared Fluorescent Dyes to Detect Proteins on Nitrocellulose Membranes (Dots)

1. Procedure for Near IR Dyes

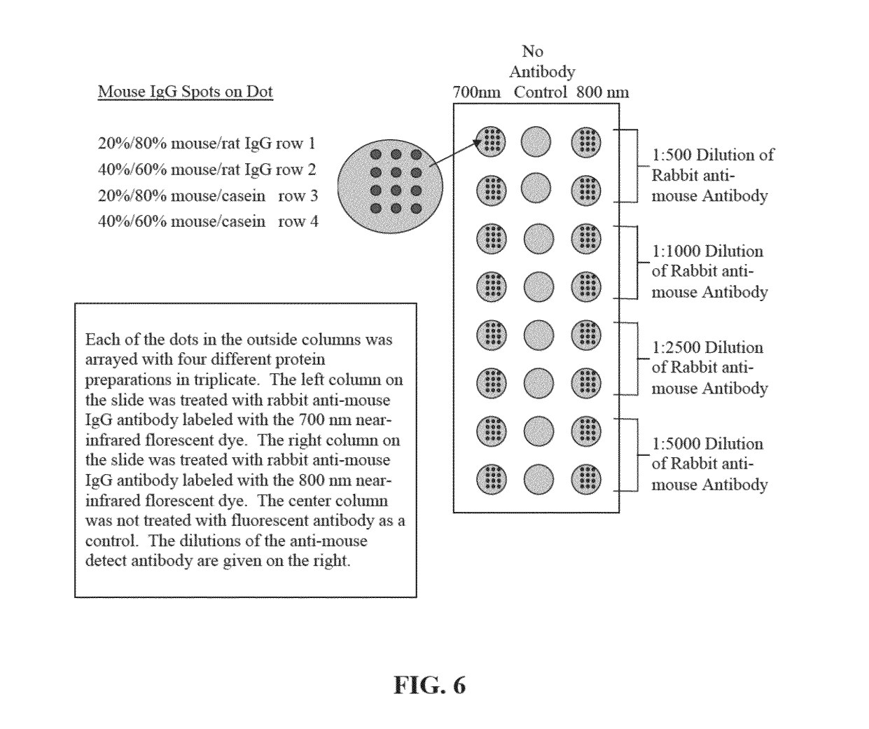

For each nitrocellulose membrane (dot), 12 protein compositions (spots) were arrayed. The compositions (spots) consisted of four different preparations of mouse IgG (Equitech-Bio, Kerrville Tex.), representing two concentrations, each performed in triplicate (FIG. 6). The spots were printed with a total protein concentration of 1 mg/mL at 20% or 40% mouse IgG in either rat IgG (Equitech-Bio, Kerrville Tex.) or bovine casein (USB Corporation, Cleveland Ohio). The membranes (dots) were blocked with a non-protein block (Pierce Biochemicals, Rockford, Ill.) for 1 hour at room temperature and incubated with a rabbit anti-mouse IgG antibody from Rockland Immunochemical, Inc. (Gilbertsville, Pa.) with either a 700 nm or an 800 nm IR Dye at one of four dilutions (1:500; 1:1000; 1:2500; or 1:5000). The slide was washed with PBST buffer, dried, and scanned using a Li-Cor IR Odyssey scanner (Li-Cor Biosciences, Lincoln, Nebr.) at 700 and 800 nm concurrently. A detailed diagram of the layout on the slide and an individual dot is shown in FIG. 6.

2. Data from the 800 nm Scan at High Resolution

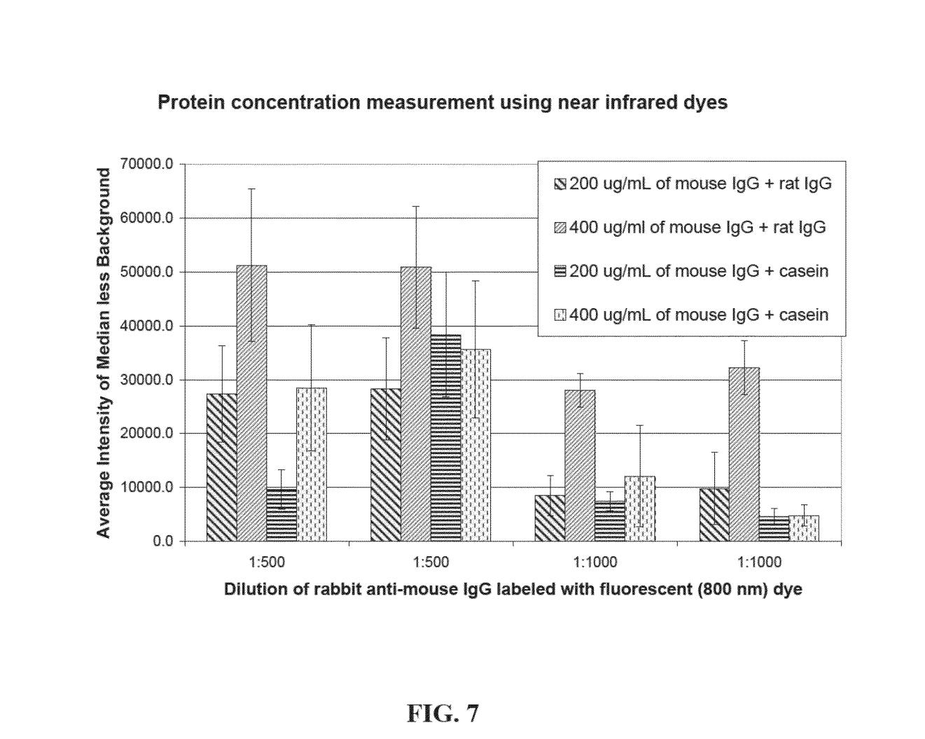

A section of one slide that included nitrocellulose membranes (dots) treated with the 800 nm dye at dilutions of 1:500 and 1:1000 was scanned at high resolution. It clearly shows the increase in signal when 400 μg/mL instead of 200 μg/mL of the mouse IgG+excess rat IgG is printed, and the increase in signal when the rabbit anti-mouse IgG antibody labeled with 800 nm dye is used at a 1:500 dilution instead of the 1:1000 dilution (FIG. 7).

3. Comparison of the 700 and 800 nm Fluorescent Scans at Low Resolution

A comparison was done by analyzing the entire nitrocellulose membrane (dot) instead of the individual spots, or arrayed proteins on each membrane, to compare the 700 and 800 nm dyes. This was performed because the Li-Cor instrument was too slow to scan a large image at the highest resolution. Therefore, a lower resolution setting was chosen and the entire fluorescence intensity of a nitrocellulose membrane (dot) was compared.

The background from the nitrocellulose membrane (dot) was determined by measuring the relative background associated with the membranes (dots). A row of eight membranes (dots) was left untreated and the median fluorescence and average of those values is listed below. The measurements were done on the same nitrocellulose membranes (dots) that were treated with both the 800 and the 700 fluorescent dyes. The data presented in Table V shows that the membranes (dots) have a much lower background at 800 nm than at 700 nm.

TABLE V
Background intensity at 700 nm and 800 nm

| | Background | Background |
|---|---|---|
| Nitrocellulose | Intensity at | Intensity at |
| Dot # | 700 nm | 800 nm |
| 1 | 497 | 235 |
| 2 | 468 | 329 |
| 3 | 440 | 182 |
| 4 | 426 | 151 |
| 5 | 422 | 96 |
| 6 | 436 | 92 |
| 7 | 457 | 99 |
| 8 | 493 | 111 |
| | Average = 455 | Average = 162 |

The detection of the mouse IgG on comparable spots is provided in Table VI. The background is 455 intensity units for the 700 nm scan and 162 intensity units for the 800 nm scan as described in Table VI. These background numbers are subtracted in the numbers below, but they do indicate that the intensity of the membranes (dots) is well over background with the 1:500 and 1:1000 dilutions.

TABLE VI
Average intensity of the 700 nM and 800 nM IRDye labeled antibody

| | Average | | Average | |
|---|---|---|---|---|
| | intensity | | intensity | |
| | of two dots | Signal/ | of two dots | Signal/ |
| | incubated | Background | incubated | Background |
| | with 700 nm | for 700 nm | with 800 nm | for 800 nm |
| | IRDye | fluorescent | IRDye | fluorescent |
| Antibody | labeled | dye | labeled | dye |
| Dilution | antibody | detection | antibody | detection |
| 500 | 3954 | 8.69 | 26928 | 166.22 |
| 1000 | 2251 | 4.95 | 24297 | 149.98 |
| 2500 | 581 | 1.28 | 1026 | 6.33 |
| 5000 | 567 | 1.25 | 337 | 2.08 |

### Example 5

### Protein Binding Capacity of Nitrocellulose Membranes (Dots)

1. Arraying (Printing) of Mouse IgG onto White Nitrocellulose Membranes (Dots)

Mouse IgG (Equitech-Bio, Kerrville Tex.) was diluted in phosphate buffered saline to eight different concentrations. Each concentration was printed six times on three different nitrocellulose membranes (dots). The dilutions were printed (arrayed) with the lowest concentration to the highest concentration and each membrane (dot) is represented spatially in Table VII. The slide was allowed to dry for 10 minutes in high humidity and then each individual dot was blocked with non-protein block (Pierce Biochemicals, Rockford, Ill.) for one hour at room temperature in a humidity chamber. After the hour the excess block was aspirated off and the dots dried.

TABLE VII
Eight concentrations of mouse IgG spotted in triplicate
Mouse Immunoglobulin G (IgG) Dilutions
One concentration was spotted 6 times on three nitrocellulose dots.

| Replicate 1 | Replicate 2 | Replicate 3 |
|---|---|---|
| 12.5 μg/mL | 12.5 μg/mL | 12.5 μg/mL |
| 25 μg/mL | 25 μg/mL | 25 μg/mL |
| 50 μg/mL | 50 μg/mL | 50 μg/mL |
| 100 μg/mL | 100 μg/mL | 100 μg/mL |
| 200 μg/mL | 200 μg/mL | 200 μg/mL |
| 300 μg/mL | 300 μg/mL | 300 μg/mL |
| 400 μg/mL | 400 μg/mL | 400 μg/mL |
| 500 μg/mL | 500 μg/mL | 500 μg/mL |

2. Incubation with Anti-Mouse IgG HRP Antibody and TMB Substrate

The dots on the slide were incubated with a 1:1000 dilution of anti-mouse IgG HRP (Santa Cruz Biotechnology, Santa Cruz Calif.) diluted in PBST, (0.1% Tween) for one hour in high humidity. The solutions were then aspirated off and the slide was washed three times for 20 minute with PBST followed by 4 washes (15 seconds each) with deionized water. The slide then was immediately incubated with TMB (Promega Corporation, Madison Wis.) solution for 2 minutes and then rinsed several times with deionized water and dried in a particle free hood.

3. Scanning of Slide and Manipulation of Images

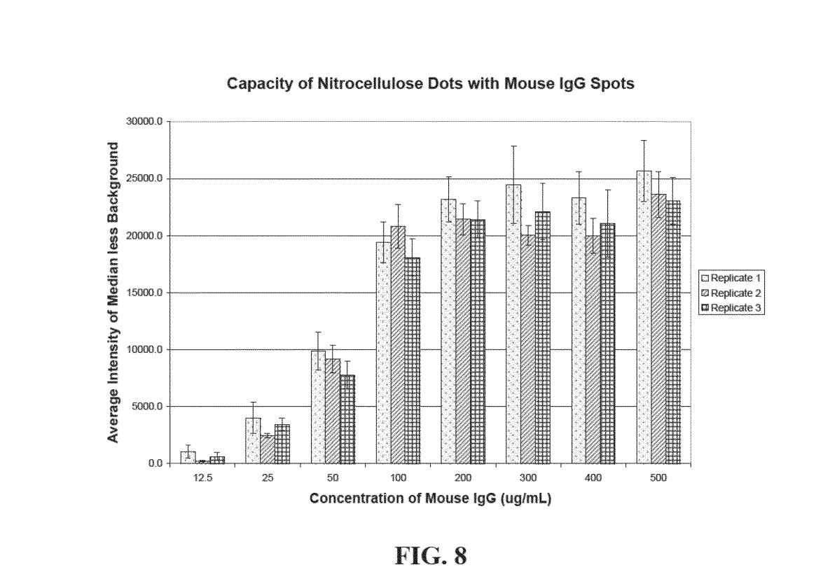

Both the front and back of the slides were scanned with a High Resolution scanner (Epson America, Long Beach, Calif.) at 2400 dpi in 16-bit grayscale file. The image was inverted using Photoshop Elements (Adobe Systems Inc., San Jose, Calif.) and saved as a TIFF file labeled with an additional “INVERTED.” The front of the slide image was analyzed with the GenePix Pro 6.0 software (Molecular Devices, Sunnyvale, Calif.) and interpreted with Microsoft Excel spreadsheet software (Microsoft Corporation, Redmond, Wash.). The capacity of the nitrocellulose membrane (dots) is depicted in FIG. 8. For all three triplicates, the maximum protein binding capacity was reached at approximately 200 μg/ml. Any further increase in the protein concentration that was arrayed did not produce any increased binding. This binding capacity (white porous nitrocellulose spots) shows significantly higher protein binding capacity than thin, optically clear nitrocellulose surfaces.

### Example 6

### Colorimetric Detection Using Supernatants from Mouse Hybridomas

In this Example, mice were immunized with small molecules conjugated to protein to create hybridomas secreting monoclonal antibodies. After the cell fusions between the mouse spleen cells and the myeloma cells, the cells were expanded and subcloned. The supernatant from these subcloned cells were screened using colorimetric detection to identify antibodies that bound to S-adenosyl homocysteine (SAH) but did not bind to SAM (S-adenosylmethionine), the carrier proteins, or the chemical linkers.

In this antigen array, seven protein samples were arrayed in 500 μM spots on nitrocellulose membranes (dots) using a Calligrapher Arrayer (BioRad, Hercules, Calif.). The arrayed proteins were detected using a colorimetric assay using horse radish peroxidase labeled goat anti-mouse antibody.

1. Printing of SAM and SAH Conjugates

Seven different antigens were printed in triplicate on each nitrocellulose membrane (dot) including: SAM-Z-bovine serum albumin (BSA), SAH-Z-BSA, SAM-G-Ovalbumin, SAH-G-Ovalbumin, BSA, Ovalbumin, and mouse IgG (Equitech-Bio, Kerrville Tex.). The Z and G denote different chemical linkers used in the conjugation reactions. The mouse IgG is used as a positive control for each dot and should bind the goat anti-mouse IgG alkaline phosphatase directly. The BSA conjugates were printed at 18 ug/mL in 100 mM carbonate buffer, pH 9.0, the ovalbumin conjugates were printed at 20 ug/mL in water, and the mouse IgG was printed at 10 ug/mL in 10 mM MOPS is 5% trehalose. The array was allowed to dry in high humidity for 20 minutes and the blocked with a non-protein block (Pierce Biochemicals, Rockford, Ill.) for 1 hour. The excess block solution was aspirated off and the array was stored overnight in an airtight container.

2. Addition of Mouse Hybridoma Supernates, Goat Anti-Mouse Alkaline Phosphates Conjugated Antibody, and BCIP/NBT

Fifteen microliters of a mouse hybridoma cell supernatant were pipetted on to each arrayed nitrocellulose membrane (dot) using Biomek 2000 pipetting station (Beckman, Fullerton, Calif.). The slide was incubated for 1 hour in a high humidity chamber. The array was then washed three times with PBST, 0.05% Tween 20. The entire slide was then incubated with a 1 in 4000 dilution of the goat anti-mouse alkaline phosphatase conjugated antibody (Sigma Chemical, St. Louis Mo.) diluted in 1 mg/mL casein (USB Corporation, Cleveland Ohio) in PBST, 0.05% Tween 20 and placed on a shaker for one hour. It was again washed three times with PBST, 0.05% Tween 20 and several times with water. The entire slide was then incubated with the BCIP/NBT substrate (Sigma Chemical, St. Louis Mo.), stopped by washing it with de-ionized ultra pure water, and dried in a clean, sterile hood.

3. Scanning and Analyzing the Array

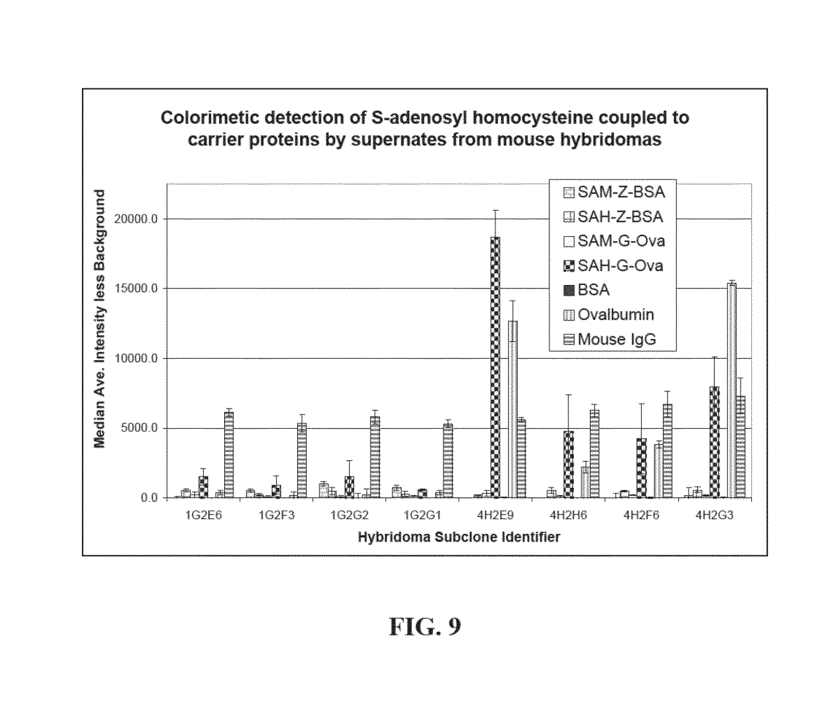

The dried slide was scanned with a High Resolution scanner (Epson America, Long Beach, Calif.) at 2400 dpi in 16-bit grayscale file. The image was inverted using Photoshop Elements (Adobe Systems Inc., San Jose, Calif.) and saved as a TIFF file labeled with an additional “INVERTED.” The image was analyzed with the GenePix Pro 6.1 software (Molecular Devices, Sunnyvale, Calif.) and interpreted with Microsoft Excel spreadsheet software (Microsoft Corporation, Redmond, Wash.). The data is reported in FIG. 9, and clearly demonstrates that the protein spots on nitrocellulose dots can be used to screen antibody binding in hybridoma bulk culture supernates, which is a very challenging mixture of cellular debris with unknown antibody identity and concentration. As would be expected, some of the antibodies recognize the desired antigen (1G2G2 and 4H2H6) more strongly than the ovalbumin carrier and some antibodies (H2G3) appear to recognize the ovalbumin carrier more strongly.

### Example 7

### High Density Arraying on Nitrocellulose Surface

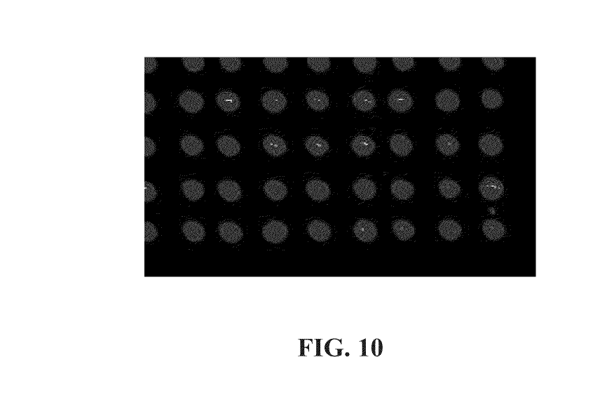

To demonstrate that small spots of protein can be arrayed on nitrocellulose prepared on a hydrophobic surface, a thin film of nitrocellulose was prepared on a glass slide as per Example 1 (FIG. 10). Replicate spots of 100 μg/mL mouse IgG (Equitech-Bio, Kerrville Tex.) in PBS were printed with the Sonoplot Arrayer (Sonoplot, Middleton Wis.) which printed 100 μm spots. The slide was blocked with 50 mL of 10 mg/mL BSA (Sigma Chemical, St. Louis Mo.) in PBS for one hour. The slide was washed three times with PBST and then incubated with 1 μg/mL goat anti-mouse IgG labeled with DyLight 647 (Pierce Biosciences, Rockford Ill.) in PBS for one hour. The slide was scanned using a GenePix 4000B scanner (Molecular Devices, Sunnyvale Calif.) at 635 nm excitation and analyzed with the bundled GenePix Pro 6.0 software. Four different rows of eight spots were analyzed and the data was interpreted with Excel Spreadsheet (Microsoft, Redwood Wash.) and reported in Table VIII.

TABLE VIII
Average intensity of arrays on nitrocellulose surface

| | Average of | | |
|---|---|---|---|
| | Median | Standard | Coefficient of |
| Row | Intensities | Deviation | Variance (%) |
| 1 | 6743.9 | 398.5 | 5.9 |
| 2 | 6164.3 | 963.7 | 15.6 |
| 3 | 6527.3 | 853.9 | 13.1 |
| 4 | 6115.1 | 497.1 | 8.1 |
| All Spots | 6387.6 | 732.0 | 11.5 |

### Example 8

### Sandwich ELISA on the Frameless Spots on Dots (Membranes)

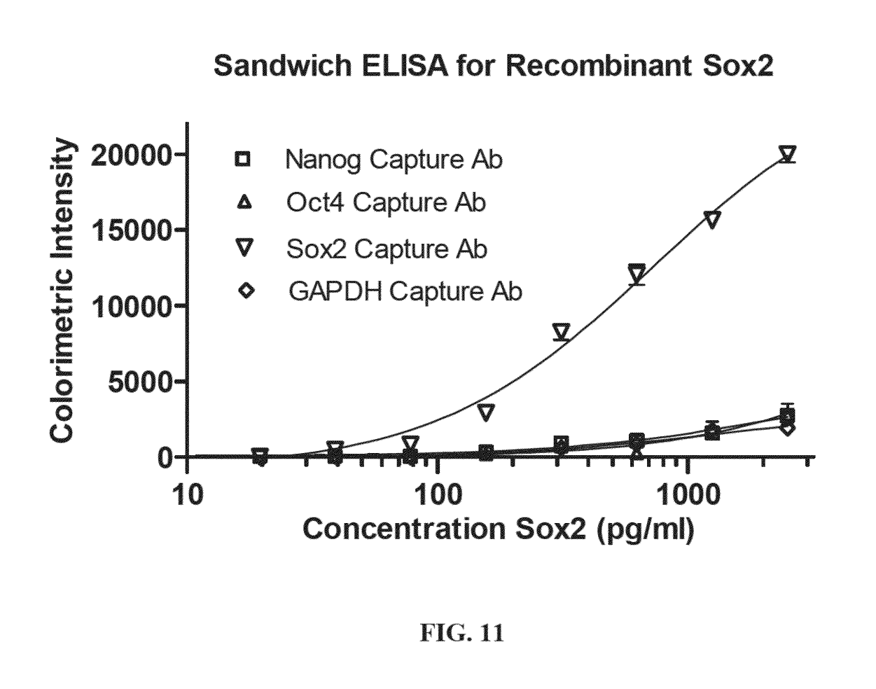

A colorimetric sandwich ELISA was performed to determine the sensitivity of the frameless nitrocellulose array (FIG. 11). Four capture antibodies for Nanog, Oct4, Sox2, and GAPDH were each printed in triplicate at 200 μg/mL on membranes. The entire array was blocked with Pierce Non-Protein block for 1 h, given 3×30-s rinses with PBST, 0.05% Tween-20 and 2×30-s rinses with ultra-pure water, then allowed to dry in a particle-free hood. Two rows of dots were given an additional block by placing 6 μL of 10 mg/mL BSA in PBS on 24 dots for 1 h in a high-humidity chamber. These dots were given 3×30-s washes with PBST, rinsed with water, and dried. A dilution series of refolded, purified Sox2 protein from E. coli inclusion bodies was made starting at 20 ng/mL and diluting 1:1 10 times for 11 dilutions of protein and one blank. All solutions were prepared in 1 mg/mL BSA in PBS. Five μL of each Sox2 dilution were incubated for 1 h at room temp in a high-humidity chamber. The excess was aspirated off quickly and the entire sheet given 3×30-s submerges in PBST. After a 1:1000 dilution in 1 mg/mL BSA in PBS, 5 μL of biotinylated Sox2 detection antibody was placed on each dot, incubated and washed the same as for the Sox2 protein. Then, 5 μL of alkaline phosphatase streptavidin, diluted to 1 μg/mL in 1 mg/mL BSA, was placed on each dot and incubated for 30 min. in a high-humidity chamber at room temperature. The array was given 5×1-min washes in PBST, 0.05% Tween-20 and rinsed once with ultra-pure water. The array then was incubated with Moss BCIP/NBT Plus until the signal could be detected ('-15 min), because determining the sensitivity of the sandwich assay on the membranes was a main goal of this experiment. This caused significant background to form on the higher concentrations of Sox2. The limit of detection was 31.6 pg/mL and the limit of quantitation was 40.7 pg/mL for Sox2 protein.

### Example 9

### Stability of Fluid Samples on White and Transparent Nitrocellulose Membranes in the Frameless Arrays

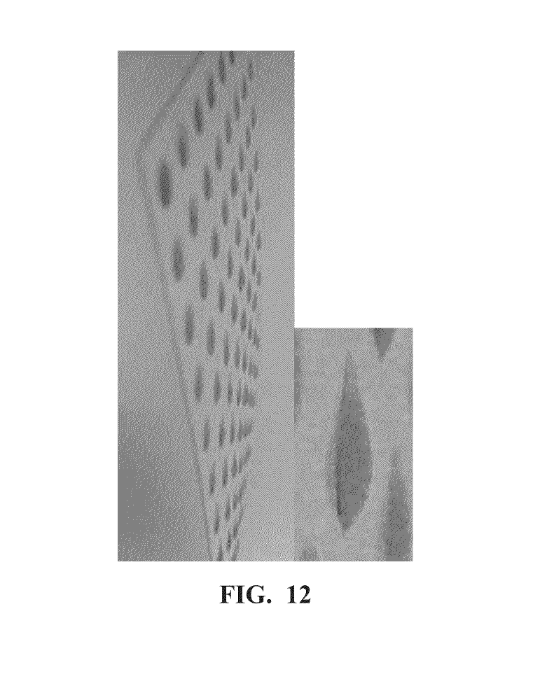

To demonstrate the stability and isolation of fluid samples on grids of nitrocellulose membranes and to show that contamination between membranes is hindered, 96 white nitrocellulose membranes were treated with fluid and analyzed. Six microliters of a solution of Protein Free Blocking Buffer (Tris) with approximately 0.01% FD&C No. 3 green dye (Fisher Scientific, Chicago, Ill.) was pipetted on to each nitrocellulose membrane. The 96 membrane array was incubated in a humidity chamber for 2 hours and removed for analysis. Each of the applied fluid samples remained on the appropriate membrane. The entire array was turned perpendicular and photographed to show that even when the array is sideways, the fluid samples remain in the appropriate position (FIG. 12). Additional experiments have been performed with various buffers, protein samples, serum samples, and cellular extracts and produce the same result; the nitrocellulose membranes maintain the fluid samples and do not allow sample contamination between membranes (data not shown).

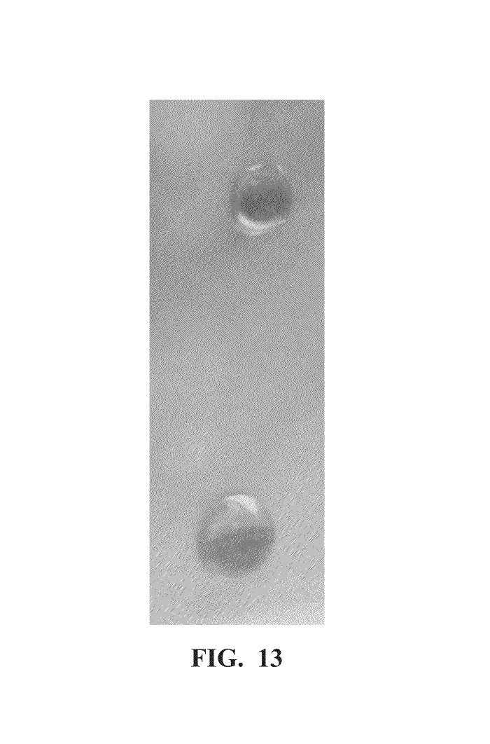

A 3% transparent nitrocellulose membrane was tested for its ability to maintain a fluid sample within the perimeter of the membrane for a frameless array application. A small buffer sample (about 5-6 μl) containing approximately 0.01% FD&C No. 3 green dye was placed on a 4 millimeter diameter transparent membrane. The substrate was moved perpendicular and photographed (FIG. 13). The fluid sample remained in position, even when tuned sides.

### Example 10

### Protein Detection Using a Transparent Nitrocellulose Surface

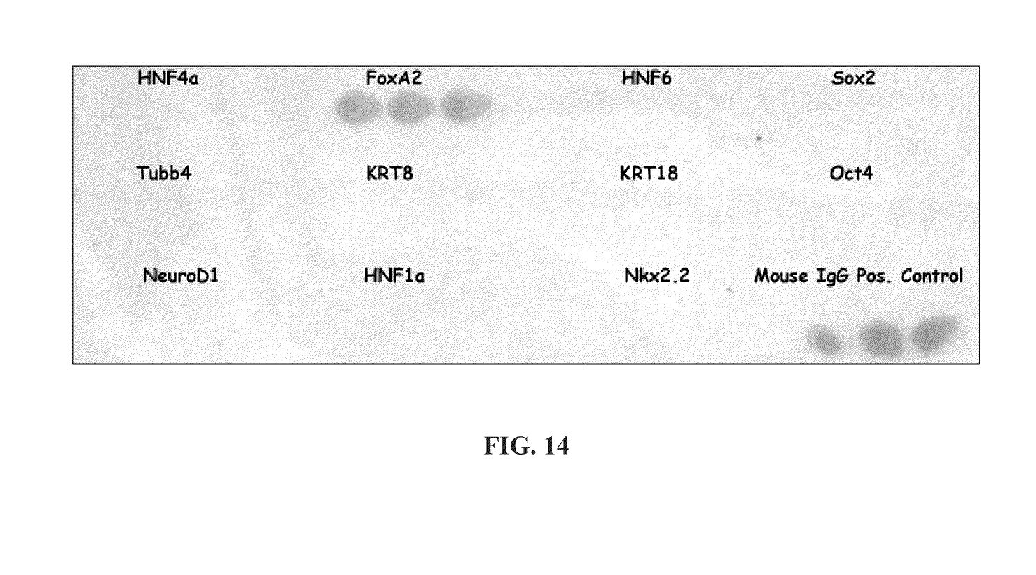

Example 9 demonstrated that both white and transparent nitrocellulose compositions can maintain fluid samples within the perimeter of the nitrocellulose membranes. In this example, proteins were arrayed on a transparent nitrocellulose surface and detected colorimetrically. Twelve human recombinant proteins, HNF4a, FoxA1, HNF6, Sox2, Tubb4, KRT8, KRT18, Oct4, NeuroD1, HNF1, Nkx2.2 and mouse IgG were printed on transparent nitrocellulose with a Calligrapher pin printer. The concentrations of each protein were approximately 1.0 mg/ml in phosphate buffered saline. After printing and drying, the array was blocked with NAP buffer diluted 1:1 in PBST buffer for 1 hour. The array then was washed three times for 10 minutes each in PBST buffer with shaking. Anti-FoxA1 antibody, 0.5 μg in 100 μl of PBST, was added to the array for 1 hour. The array was then washed as previously described and incubated with alkaline-phosphatase conjugated goat-anti-mouse IgG for 1 hour with shaking. The array was washed as before with PBST, rinsed briefly two times with 20 mM phosphate buffer pH 7.4. The array was then incubated with MOSS alkaline phosphatase substrate until the dark spots were visible (approximately 15 minutes). The image was scanned using an Epson V700 scanner. FIG. 14 is a representation of the scan, which depicts the results. The anti-FoxA2 antibody produced a very strong colorimetric signal, while the other human protein targets were not detected. The positive control mouse IgG also gave a very strong signal. The array contained twelve recombinant proteins, but only FoxA2 was detected by the anti-FoxA2 antibody. This experiment clearly demonstrates that transparent nitrocellulose composition maintains the sample within the perimeter, and is a suitable membrane for the array. In addition, this experiment demonstrates that the array can comprise numerous analytes and still produce specific and sensitive results in a high-throughput fashion.

Although the invention has been described in considerable detail by the preceding specification, this detail is for the purpose of illustration and is not to be construed as a limitation upon the following appended claims. All cited reports, references, U.S. patents, allowed U.S. patent applications, and U.S. patent Application Publications are incorporated herein by reference.

What is claimed is:

1. A method for producing a frameless array comprising:
coupling at least two segregated membranes to a substrate, wherein said membranes comprise a composition comprising nitrocellulose, and further wherein said composition is formulated to maintain an applied sample on the membrane; and
coupling an analyte to said membranes to produce a frameless array.

2. The method of claim 1, wherein the substrate is selected from the group consisting of glass, polyester, polyethylene, and polypropylene.

3. The method of claim 1, wherein said nitrocellulose is selected from the group consisting of opaque, translucent, and transparent.

4. The method of claim 1, wherein said composition comprises cellulose acetate and a solvent.

5. The method of claim 1, wherein said formulation comprises a solvent selected from the group consisting of acetone, ethanol, amyl acetate, and butanol.

6. The method of claim 1, wherein said formulation comprises a solvent mixture comprising acetone, ethanol, and butanol.

7. The method of claim 1, wherein the segregated membrane is aligned in a grid selected from the group consisting of an 8×12 grid, a 16×24 grid, and a 32×48 grid.

8. The method of claim 1, wherein each segregated membrane has an area of about 28 square mm.

9. The method of claim 1, wherein each segregated membrane has an area of about 7 square mm.

10. The method of claim 1, wherein each segregated membrane has an area of about 1 square mm.

11. The method of claim 1, wherein the top of the membrane is the highest point on said frameless array.

12. The method of claim 1, wherein said analyte is selected from the group consisting of: a probe, RNA, DNA, a peptide, a fragment of a protein, an antibody, and a protein.

13. The method of claim 1, wherein said composition is formulated to maintain an applied fluid within the perimeter of the membrane for a period of time selected from the group consisting of 0.1-0.5, 0.51-1.0, 1.1-2.0, 2.1-4.0, 4.1-6.0, 6.1-8, 8.1-10, 10.1-12, 12.1-16, 16.1-20, 20.1-24, 24.1-30, 30.1-36, 36.1-48, 48.1-54, 54.1-60, 60.1-72, 72.1-96, and 96.1-120 hours.

14. A frameless array comprising: (a) at least two segregated membranes coupled to a substrate, wherein said membranes comprise a composition comprising nitrocellulose, and further wherein said composition is formulated to maintain an applied fluid within the perimeter of the membrane and (b) an analyte coupled to said membranes.

15. The array of claim 14, wherein the substrate is selected from the group consisting of glass, polyester, polyethylene, and polypropylene.

16. The array of claim 14, wherein said nitrocellulose is selected from the group consisting of opaque, translucent, and transparent.

17. The array of claim 14, wherein said composition comprises cellulose acetate.

18. The array of claim 17, wherein said composition further comprises a solvent selected from the group consisting of acetone, ethanol, amyl acetate, and butanol.

19. The array of claim 14, wherein the segregated membrane is aligned in a grid selected from the group consisting of an 8×12 grid, a 16×24 grid, and a 32×48 grid.

20. The array of claim 14, wherein each segregated membrane has an area selected from the group consisting of: about 1, 7, and 28 square mm.
